(19) United States
(12) Patent Application Publication
(10) Pub. No.: US 2023/0277220 A1
(43) Pub. Date: Sep. 7, 2023

(54) DETACHABLE MOTOR

(71) Applicant: ORTHOSPIN LTD., Yokneam Illit (IL)

(72) Inventors: Shahar HARARI, Tel-Aviv (IL); Oren COHEN, Moreshet (IL)

(21) Appl. No.: 18/018,498

(22) PCT Filed: Jul. 29, 2021
(86) PCT No.: PCT/IL2021/050922

§ 371 Date: Jan. 27, 2023

Related U.S. Application Data
(60) Provisional application No. 63/058,686, filed on Jul. 30, 2020.

(51) Int. Cl. A61B 17/66; A61B 17/62; A61B 90/98

(52) U.S. Cl. CPC A61B 17/66; A61B 17/62; A61B 90/98; A61B 2017/00221

(57) ABSTRACT

A kit, including: a strut of a bone fixation device including a fixed portion and an extending portion, wherein the strut comprises a linear actuator mechanically connected to the extending portion; at least one motor adaptor coupled to the linear actuator, wherein the motor adaptor comprises a motor fastener; at least one motor unit selectively attachable and detachable from the motor fastener, wherein the motor unit is configured to functionally couple to the linear actuator and axially extend the extending portion of the strut; wherein the motor fastener is shaped and sized to receive a portion of the motor unit.

### RELATED APPLICATION/S

This application claims the benefit of priority under 35 USC § 119(e) of U.S. Provisional Patent Application No. 63/058,686 filed 30 Jul. 2020, the contents of which are incorporated herein by reference in their entirety.

### FIELD AND BACKGROUND OF THE INVENTION

The present invention, in some embodiments thereof, relates to a detachable motor and, more particularly, but not exclusively, to a detachable motor of a bone fixation device.

### SUMMARY OF THE INVENTION

Some examples of some embodiments of the invention are listed below. Features from one example may be combined with features from other examples:

Example 1. A kit, comprising:
a strut of a bone fixation device including a fixed portion and an extending portion, wherein said strut comprises a linear actuator mechanically connected to said extending portion;
at least one motor adaptor coupled to said linear actuator, wherein said motor adaptor comprises a motor fastener;
at least one motor unit selectively attachable and detachable from said motor fastener, wherein said motor unit is configured to functionally couple to said linear actuator and axially extend said extending portion of said strut;
wherein said motor fastener is shaped and sized to receive a portion of said motor unit.
   Example 2. A kit according to example 1, wherein said motor fastener is shaped and sized to receive an end of said motor unit, and to restrain lateral movement of said motor unit end.
   Example 3. A kit according to any one of examples 1 or 2, wherein said motor fastener comprises a socket shaped to receive said motor end.
   Example 4. A kit according to example 3, wherein said motor unit comprises housing, a motor and a gear extending from said motor unit within said housing, and wherein said motor unit end comprises a portion of said gear extending from said housing.
   Example 5. A kit according to example 4, wherein said portion of said gear extending from said housing has a smaller diameter compared to a diameter of said motor housing.
   Example 6. A kit according to any one of examples 3 to 5, wherein said motor end is a conical motor end and/or a tapered motor end shaped to be positioned within said socket.
   Example 7. A kit according to any one of the previous examples, wherein said strut comprises one or more radially extending portions that interface with said motor adaptor.
   Example 8. A kit according to any one of the previous examples, wherein said motor adaptor comprises housing with one or more openings, and wherein said housing is attached to the strut by one or more or screws or pins.
   Example 9. A kit according to any one of examples 1 to 7, wherein said motor adaptor comprises housing with one or more openings shaped to receive a strut, wherein an inner diameter of said openings is larger than an outer diameter of said strut.
   Example 10. A kit according to example 9, wherein said one or more openings forms a channel sized and shaped to receive said strut.
   Example 11. A kit according to any one of examples 9 or 10, wherein an inner portion of said one or more openings is round.
   Example 12. A kit according to any one of examples 9 to 11, wherein said strut comprises a window, and wherein said motor adaptor when coupled to said strut does not block said window.
   Example 13. A kit according to any one of examples 9 to 12, wherein said strut comprises a visual indicator indicating an extending length of the strut, and wherein said motor adaptor housing comprises a window or one or more elongated openings, at least partly aligned with said visual indicator when said motor adaptor is coupled to said strut.
   Example 14. A kit according to any one of examples 9 to 13, comprising at least one motor connector shaped and sized to fasten said motor unit to said housing of said motor adaptor.
   Example 15. A kit according to example 14, wherein said motor connector comprises one or more protrusions configured to fit into openings in said housing and to lock said motor connector to said housing.
   Example 16. A kit according to any one of examples 14 or 15, wherein said motor unit comprises a groove, and wherein said motor connector is shaped and sized to fit into said groove when fastening said motor unit to said motor adaptor housing.
   Example 17. A kit according to any one of examples 14 to 16, wherein said motor connector comprises a clip.
   Example 18. A kit according to any one of the previous examples, wherein said strut comprises a gear of said linear actuator, located near said extending portion of said strut.
   Example 19. A kit according to example 18, wherein said linear actuator gear is located at a distance of up to 5 cm from said extending portion.
   Example 20. A kit according to any one of examples 18 or 19, wherein said motor adaptor comprises a gear, and wherein said motor adaptor gear is configured to interlock with said linear actuator gear when said motor adaptor is coupled to the strut, such that rotation of said motor adaptor gear axially moves said linear actuator.
   Example 21. A kit according to example 20, wherein said motor end interacts with said motor adaptor gear when said motor is selectively attached to the motor adaptor.
   Example 22. A kit according to example 21, comprising a manual motor adaptor interface shaped and sized to interlock with said motor adaptor gear such that manual rotation of said manual motor adaptor interface axially moves said linear actuator.
   Example 23. A kit according to example 22, wherein said motor adaptor gear alternately interlocks with said manual motor adaptor interface and said motor end.
   Example 24. A kit according to any one of examples 22 or 23, wherein an end of said manual motor adaptor interface is shaped to be positioned within said motor adaptor fastener.
   Example 25. A kit according to any one of examples 20 to 24, comprising at least one gear lock, attachable and detachable from said motor adaptor, and configured to interlock and stop a movement of said motor adaptor gear.
   Example 26. A kit according to example 25, wherein said at least one gear lock comprises a first end shaped and size to be positioned within said motor fastener and to interlock with said motor adaptor gear, and a second end shaped and sized to extend out from said motor fastener and to interlock with a housing of said motor adaptor.
   Example 27. A kit according to any one of the previous examples comprising a bone fixation device which includes said strut and at least two spaced apart frames, wherein each frame is configured to be coupled to a different end of said strut, and to a bone connector extending from a bone.
   Example 28. A kit according to example 27, wherein at least one of said spaced apart frames comprises an arc or a ring, surrounding at least partly a limb of a patient.
   Example 29. A kit according to any one of examples 27 or 28, comprising:
at least one electric cable; and
a control unit reversibly coupled to a frame of said at least two frames, wherein said control unit is connected to said motor and/or said motor adaptor by said at least one electric cable.
   Example 30. A kit according to example 29, comprising a control unit frame interface, fixedly connectable to said frame of said at least two frames, and wherein said control unit is configured to be attachable and detachable from said control unit frame interface.
   Example 31. A kit according to example 30, wherein said control unit and/or said control unit frame interface comprise a snap fit lock or an interference lock configured to allow attachment and detachment of said control unit from said control unit frame interface.
   Example 32. A kit according to any one of examples 29 to 31, comprising one or more cable splitter boxes configured to be fixedly attached to a frame of the bone fixation device and to combine at least two cables extending from two different motors into a single cable connected to said control unit.
   Example 33. A kit according to any one of examples 29 to 32, comprising one or more cable fasteners configured to be fixedly attached to a frame of the bone fixation device, and to fasten said at least one cable to said bone fixation device.
   Example 34. A kit according to any one of examples 29 to 33, comprising at least one cable wrapper, configured to be fixedly attached to a frame of the bone fixation device, and to fasten a loose portion of said at least one cable.
   Example 35. A kit according to any one of examples 29 to 34, wherein said control unit comprises at least one motor connector configured to receive said at least one cable.
   Example 36. A kit according to example 35, comprising a control circuitry connected to said at least one motor connector, and a user interface configured to generate a human detectable indication, wherein said control circuitry signals said user interface to generate said human detectable indication according to signals received from said at least one motor connector.
   Example 37. A kit according to example 36, wherein said at least one motor unit comprises at least one electric motor and at least one positioning sensor configured to record rotation of said at least one electric motor, and wherein said control circuitry measures an extension of a strut coupled to said at least one motor unit using said motor rotation recordings of said at least one positioning sensor.
   Example 38. A kit according to any one of the previous examples, wherein said linear actuator is a non-motorized mechanical linear actuator.
   Example 39. A motor adaptor coupled to a strut of a bone fixation device and selectively coupled to a motor unit, comprising:
housing coupled to said strut of a bone fixation device;
a motor fastener in said housing shaped and sized to receive and restrain lateral movement of an end of said motor unit.
   Example 40. An adaptor according to example 39, wherein said housing comprises at least one opening shaped and sized to receive said strut.
   Example 41. An adaptor according to any one of examples 39 or 40, comprising one or more connectors and/or openings in said housing configured to attach the motor adaptor to a strut of a bone fixation device using one or more pins or screws crossing said openings.
   Example 42. An adaptor according to any one of examples 39 to 41, comprising a gear in said housing positioned to interact with a gear of a linear actuator of said strut when said housing is coupled to said strut.
   Example 43. An adaptor according to claim example 42, wherein said motor adaptor gear is located at said motor fastener, and is configured to interlock with a motor end.
   Example 44. An adaptor according to any one of examples 42 or 43, comprising a motor adaptor manual interface configured to penetrate at least partly into said housing and interlock with said motor adaptor gear.
   Example 45. An adaptor according to example 44, wherein said motor adaptor gear is configured to alternately interlock with said motor end and said motor adaptor manual interface.
   Example 46. A method for coupling a motor to a bone fixation device, comprising: coupling an end of a motor into a socket of a motor adaptor connected to a strut of a bone fixation device;
restraining lateral movements of said motor end by said socket;
activating said motor to extend an extending portion of said strut.
   Example 47. A method according to example 46, wherein said coupling comprises functionally coupling said motor end with a linear actuator gear of said strut.
   Example 48. A method according to any one of examples 46 or 47, wherein said coupling comprises interlocking said motor end with a gear of said motor adaptor.
   Example 49. A method according to any one of examples 46 to 48, comprising:
connecting said motor to a control unit of a bone fixation device, and wherein said activating comprises activating said motor by said control unit according to indications stored in a memory of said control unit.
   Example 50. A method according to any one of examples 46 to 49, comprising adjusting an angle between a horizontal axis of said motor adaptor and at least one frame of said bone fixation device, and locking said motor adaptor at said adjusted frame, prior to said activating.
   Example 51. A method according to example 50, wherein said adjusting comprising rotating said motor adaptor and said strut around a longitudinal axis of said strut.
   Example 52. A method according to example 46, comprising:
attaching said motor adaptor to a strut of a bone fixation device prior to said coupling.
   Example 53. A method according to example 52, wherein said attaching comprises functionally coupling a gear of said motor adaptor with a linear actuator of said strut.
   Example 54. A method according to any one of examples 46 to 53, comprising:
identifying that said motor is coupled to a correct strut of a bone fixation device by reading using a computer an identification code associated with said motor prior to said activating.
   Example 55. A method according to example 54, wherein said identification code comprises an RFID and wherein said computer comprises an RFID reader.
   Example 56. A method for replacing a strut of a bone fixation device, comprising:
detaching a motor from a first strut connected to a bone fixation device;
replacing in said bone fixation device said first strut with a second strut;
attaching said detached motor to said second strut.
   Example 57. A method according to example 56, wherein said detaching comprises detaching said motor from a first motor adaptor coupled to the first strut, and wherein said attaching comprises attaching sad motor to a second motor adaptor coupled to said second strut.
   Example 58. A method according to example 56, wherein said detaching comprises detaching a motor adaptor connected to said motor, from said first strut, and wherein said attaching comprises attaching said motor adaptor to said second strut.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as control and monitor the extension of each strut of a bone fixation device, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart of a general process for coupling a motor to a strut, according to some exemplary embodiments of the invention;

FIGS. 2A-2F are block diagrams showing coupling a motor to a strut by a motor adaptor, according to some exemplary embodiments of the invention;

FIG. 2G is a block diagram showing a gear lock coupled to a motor adaptor, for example when a motor is detached, according to some exemplary embodiments of the invention;

FIG. 2H is a block diagram showing connections between a motor coupled to a strut and a control unit, according to some exemplary embodiments of the invention;

FIG. 3 is a flow chart of a detailed process for coupling a motor to a strut, according to some exemplary embodiments;

FIGS. 4A-4C are schematic illustrations showing coupling of a motor to a strut comprising a motor adaptor, according to some exemplary embodiments of the invention;

FIGS. 5A-5D are schematic illustrations showing components of a motor unit and a motor adaptor coupled to a strut, according to some exemplary embodiments of the invention;

FIGS. 6A-6D are schematic illustrations of an add-on motor adaptor coupled to a strut, according to some exemplary embodiments of the invention;

FIGS. 7A-7B are schematic illustrations showing water sealing between the motor unit and the motor adaptor and water drainage, according to some exemplary embodiments of the invention;

FIGS. 7C-7E are schematic illustrations showing fitting between the motor adaptor and a motor unit coupled to the motor adaptor, and struts in various lengths, according to some exemplary embodiments of the invention;

FIGS. 7F-7K are schematic illustrations showing changing and fixing an angle between the motor adaptor a frame of a bone fixation device, according to some exemplary embodiments of the invention;

FIGS. 8A-8C are schematic illustrations showing replacement of a strut and re-using of the motor unit with the new strut, according to some exemplary embodiments of the invention;

FIGS. 9A-9G are schematic illustrations showing a manual motor adaptor interface, and interactions of the manual motor adaptor interface with a motor adaptor, according to some exemplary embodiments of the invention;

FIGS. 10A-10E are schematic illustrations showing an assembly process of a bone fixation system, and the components of the system, according to some exemplary embodiments of the invention;

FIGS. 11A-11G are schematic illustrations showing an assembly of a control unit to a bone fixation device, and connection of the control unit to detachable motor units, according to some exemplary embodiments of the invention;

FIGS. 12A-12C are schematic illustrations of a motor unit with at least one positioning sensor, according to some exemplary embodiments of the invention; and

FIGS. 13A-13F are schematic illustrations of a gear lock, and interaction of the gear lock with a motor adaptor, according to some exemplary embodiments of the invention.

### DESCRIPTION OF SPECIFIC EMBODIMENTS OF THE INVENTION

The present invention, in some embodiments thereof, relates to a detachable motor and, more particularly, but not exclusively, to a detachable motor of a bone fixation device.

A broad aspect of some embodiments relates to coupling, for example selectively coupling a motor unit to a strut, for example a strut of an orthopedic fixation device, for example an external bone fixation device.

An aspect of some embodiments relates to restraining a movement of a motor unit coupled to the strut. In some embodiments, the movement of the motor unit, for example lateral and/or axial movement, is restrained when the motor unit is coupled to the strut. In some embodiments, the movement of the motor unit relative to the strut is restrained. Optionally, the movement of the motor unit relative to a linear actuator of the strut is restrained. In some embodiments, the motor unit is a detachable motor unit, configured to attach and detach, for example selectively, from the strut.

According to some embodiments, the motor unit is selectively coupled to the strut via an adaptor, for example a motor adaptor. In some embodiments, the motor adaptor comprises at least one motor restrainer, configured to restrain the movement of the motor unit. In some embodiments, the restrainer of the motor adaptor is configured to restrain lateral and/or axial movement of the motor unit. In some embodiments, the motor unit comprises a motor, a driving shaft of the motor and a gear of the motor. In some embodiments, a gear of the motor, is selectively coupled to the linear actuator, for example to a gear of the linear actuator.

According to some embodiments, the motor unit interlocks, for example to a motor adaptor that is connected to the linear actuator. In some embodiments, a restrainer of the motor adaptor interlocks the motor unit with the linear actuator. Optionally, the restrainer of the motor adaptor interlocks a gear of the motor unit or at least one end of the motor with a gear of the linear actuator.

An aspect of some embodiments relates to using the same adaptor coupled to a strut for both manual and motorized adjustments of the strut. In some embodiments, manual-induced movement and motorized induced movement is delivered to an actuator of the strut, for example a linear actuator, through the same transmission element. In some embodiments, the transmission element is a transmission element coupled to the linear actuator, for example to a gear of the linear actuator.

According to some embodiments, the motor unit and a manual interface for delivering the manual-induced movement are connected in parallel to the same transmission element. Alternatively, the motor unit and the manual interface are interchangeable.

An aspect of some embodiments relates to delivering movement to a linear actuator of a strut near an extending portion of the strut. In some embodiments, a transmission element, for example a motorized gear, contacts the linear actuator near an extending portion of the strut, for example at a distance smaller than 10 cm, smaller than 8 cm, smaller than 5 cm, smaller than 3 cm, smaller than 2 cm or any intermediate, smaller or larger distance from an extending portion of the strut.

According to some embodiments, a motor unit is coupled to a strut, near an extending portion of the strut and distant from a fixed end of the strut, for example at a distance smaller than 10 cm, smaller than 8 cm, smaller than 5 cm, smaller than 3 cm, smaller than 2 cm or any intermediate, smaller or larger distance from an extending portion of the strut. As used herein, the term near means closer to a first location and distant from a second location. In some embodiments, a gear of the motor unit is coupled to a linear actuator of the strut at a distance smaller than 10 cm, smaller than 8 cm, smaller than 5 cm, smaller than 3 cm, smaller than 2 cm or any intermediate, smaller or larger distance from an extending portion of the strut.

An aspect of some embodiments relates to separating in time and location between a connection of an external fixation system that includes struts to a patient bone, and a coupling of a motor unit to the strut. In some embodiments, the motor unit is coupled to the strut after completing a surgery for connecting the fixation device to a bone of a patient. In some embodiments, the motor unit is coupled to the strut outside an operation room, for example at a clinic or at the patient's home.

According to some embodiments, during and/or after the surgery, a strut length is changed, for example by manual manipulation of the motor adaptor coupled to the strut, for example the gear of the motor adaptor. In some embodiments, the motor adaptor is manually manipulated by a manual interface in the motor adaptor, for example a manual interface that is interlocked with a linear actuator of the strut. In some embodiments, the manual interface is removably coupled to the motor adaptor and/or to the linear actuator. In some embodiments, selectively coupling of a motor unit to the motor adaptor decouples the manual interface from the linear actuator. Additionally or alternatively, the selectively coupling of the motor unit to the motor adaptor, releases the manual interface from the motor adaptor.

According to some embodiments, a strut connected to the bone fixation device is replaced without replacing a motor unit. In some embodiments, the same motor unit is coupled to a new strut. In some embodiments, the motor unit is detached from the strut, for example from a motor adaptor coupled to the strut, before the strut is replaced. Optionally, the motor adaptor coupled to the strut, for example fixedly coupled, is replaced with the strut.

An aspect of some embodiments relates to adjusting a relative position of strut attachments to minimize external interference during treatment. In some embodiments, the strut attachment comprise at least one motor adaptor or a motor unit coupled to the strut. Alternatively, the strut attachments comprise a motor unit coupled to the strut, for example via the motor adaptor, and at least one wire, for example an electrical wire connecting the motor unit and/or the motor adaptor to a control unit. Additionally or alternatively, the relative position of the strut attachments is adjusted according to a patient anatomy and/or location of subjects that may interfere with the treatment.

According to some embodiments, an angle between a frame, for example a ring of an external fixation device and a strut assembly comprising a strut and a motor adaptor connected to the frame, is adjusted. In some embodiments, an angle between a plane perpendicular and tangent to the ring, and a transverse axis of the strut assembly is adjusted. In some embodiments, the angle is adjusted by rotating a strut of the strut assembly around a longitudinal axis of the strut. Optionally, the angle is adjusted prior to coupling a motor unit to the motor adaptor of the strut assembly. Alternatively, the angle is adjusted when the motor unit is coupled to the motor adaptor.

According to some embodiments, the strut assembly is locked at a desired angle relative to a frame or plane perpendicular and tangent to the frame, by a lock, for example a screw or a pin that controls a rotational movement of a strut of a strut assembly around a longitudinal axis of the strut.

According to some embodiments, length and/or position of wires connecting the motor unit and/or the motor adaptor to a control unit of the bone fixation device, are adjusted. In some embodiments, the wires length and/or position are adjusted to minimize interference to the treatment, for example to minimize potential interactions between the wires and an external object. In some embodiments, the wires are attached to at least a portion of a bone fixation device, for example by a wire attachment clip. Alternatively or additionally, wires from two or more sources are attached to the bone fixation device using a wire splitter attachment. In some embodiments, wires length is adjusted by wrapping excess wire around a wire wrapper attached to the bone fixation device.

According to some exemplary embodiments a motor unit coupled to the motor adaptor comprises a gear. Optionally, the motor unit comprises an encoder. In some embodiments, the motor unit comprises a user interface configured to generate at least one human detectable indication, for example an audio and/or a visual indication. In some embodiments, the motor unit user interface generates an indication to indicate an activation status of the motor unit, for example whether a specific motor unit is activated or not. In some embodiments, the motor unit user interface generates an audio signal to indicate whether a specific motor is activated. In some embodiments, the motor unit user interface comprises at least one LED.

According to some exemplary embodiments, the motor unit comprises a water sealed housing. In some embodiments, the motor unit comprises a seal between an end of the motor unit contacting, for example interlocking, with a motor adaptor, and the motor unit housing, for example to prevent water entry to electrical circuits within the housing.

According to some embodiments, a bone fixation device comprises at least two frames, and 2 or more, for example 6 struts interconnecting the at least two frames. In some embodiments, the bone fixation device is a hexapod, and comprises 6 struts interconnecting the at least two frames. It should be also clear that a strut as used herein can be connected as a monorail to two spaced apart pins or bone connectors, connected to two portions of a bone.

According to some embodiments, a strut of a bone fixation device including a linear actuator, a motor adaptor coupled to the linear actuator, and a motor unit are provided as a kit. In some embodiments, at least one kit is provided for a bone fixation device. In some embodiments, 2, 3, 4, 5, 6, 7 or any larger number of kits is provided for a bone fixation device. In some embodiments, a hexapod bone fixation device comprises 6 kits. In some embodiments, the number of kits is determined by the number of struts included in a bone fixation device.

In some embodiments the terms motor and a motor unit which comprise the motor are interchangeable.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Process for Motor Unit Coupling

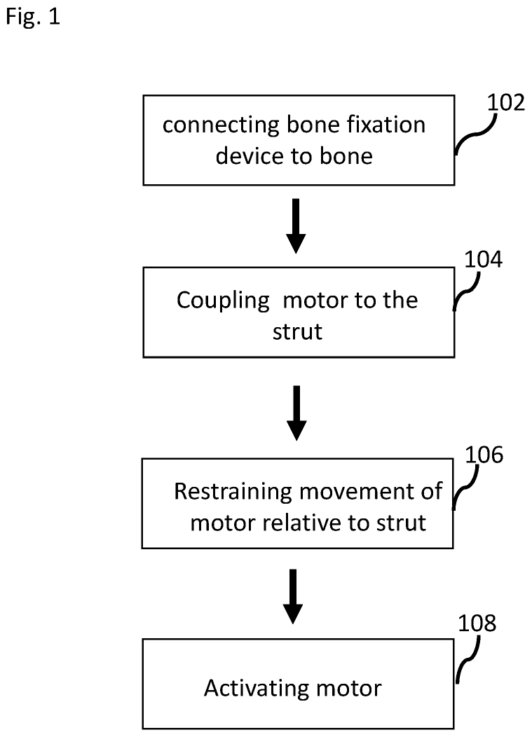

According to some exemplary embodiments, a motor is coupled to a strut, for example a strut of a bone fixation device in a separate process from connecting the bone fixation device to a patient bone. Reference is now made to FIG. 1 depicting a general process for coupling a motor to a strut of a bone fixation device, according to some exemplary embodiments of the invention.

According to some exemplary embodiments, a bone fixation device is connected to a bone of a patient, at block 102. In some embodiments, the bone fixation device comprise at least two spaced apart frames, at least one rod per frame extending from the frame into a bone portion, an one or more struts connecting the two frames. In some embodiments, each of the one or more struts comprises a linear actuator configured to change a distance between the two spaced apart frames. In some embodiments, the one or more struts are assembled between the two frames at block 102. In some embodiments, the bone fixation device comprises 4, 5, 6, 7, 8 or any smaller or larger number of struts.

According to some exemplary embodiments, the bone fixation device is connected to the bone in a surgical process, for example a surgery performed in an operating room. In some embodiments, the one or more struts are connected to the frames of the bone fixation device during the surgery, while the patient is in the operating room. In some embodiments, the one or more struts are sterilized prior to assembly to the bone fixation device. In some embodiments, at least some of the struts comprise an integral motor adaptor. In some embodiments, the struts and the motor adaptor are sterilized prior to the assembly.

According to some exemplary embodiments, a motor is coupled, for example selectively coupled to the strut at block 104. In some embodiments, the motor is coupled to the motor adaptor of the strut. Additionally, the motor is coupled to a linear actuator of the strut, for example via the motor adaptor. In some embodiments, the motor is coupled to the strut after the surgery ends. Optionally, the motor is coupled to the strut outside of the operating room, for example when the patient is at a clinic or at home.

According to some exemplary embodiments, movement of the motor relative to the strut is restrained at block 106. In some embodiments, coupling of the motor to the strut, for example to a linear actuator of the strut is restrained at block 106. In some embodiments, lateral and/or axial movement relative to the strut, for example relative to the linear actuator of the strut, is restrained at block 106. In some embodiments, the movement of the motor is restrained by the motor adaptor attached to the strut. In some embodiments, the motor adaptor restrains the movement of the motor by interlocking at least part of the motor with the strut, for example with a linear actuator of the strut.

According to some exemplary embodiments, the motor is activated at block 108. In some embodiments, the motor is activated once the movement of the motor are restrained relative to the strut, for example relative to a linear actuator of the strut. In some embodiments, the motor is activated to extend and/or to shorten a length of the linear actuator of the strut. In some embodiments, extending and/or shortening the length of the linear actuator changes the length of the strut and the distance between at least two rings of the bone fixation device. In some embodiments, the motor is activated according to a treatment plan.

According to some exemplary embodiments, the motor coupled to the linear actuator moves the linear actuator via the motor adaptor of the strut. In some embodiments, the motor moves the linear actuator using a gear of the motor adaptor connected to the linear actuator, for example to a gear of the linear actuator. Alternatively, the motor adaptor interlocks an end of the motor, for example an end of the motor including a gear with a gear of the linear actuator. In some embodiments, interlocking the motor end with the linear actuator gear allows, for example, direct interaction between the motor and the linear actuator.

### Exemplary Strut Assembly and Kit

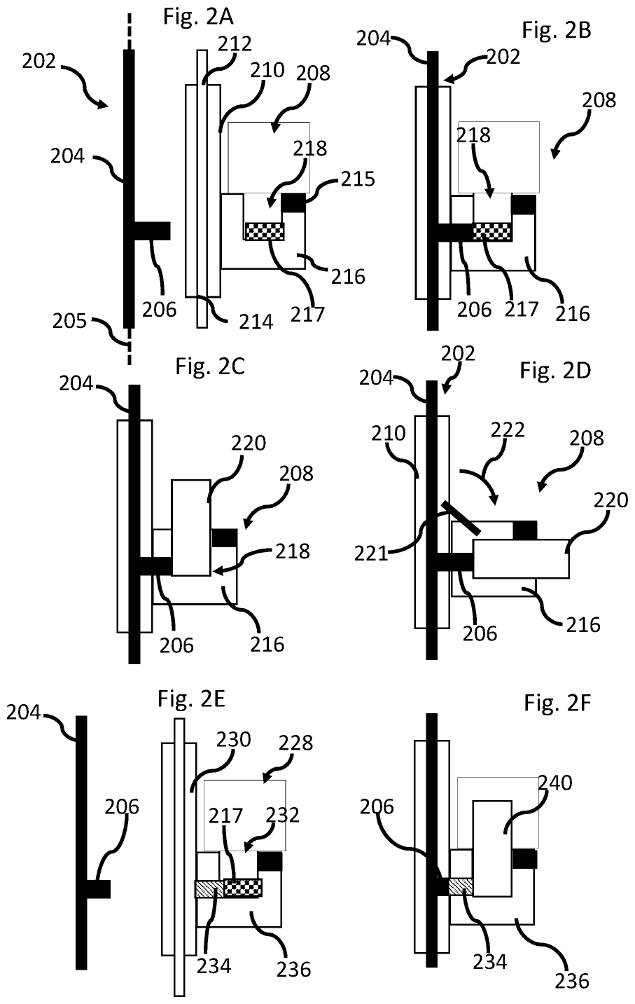

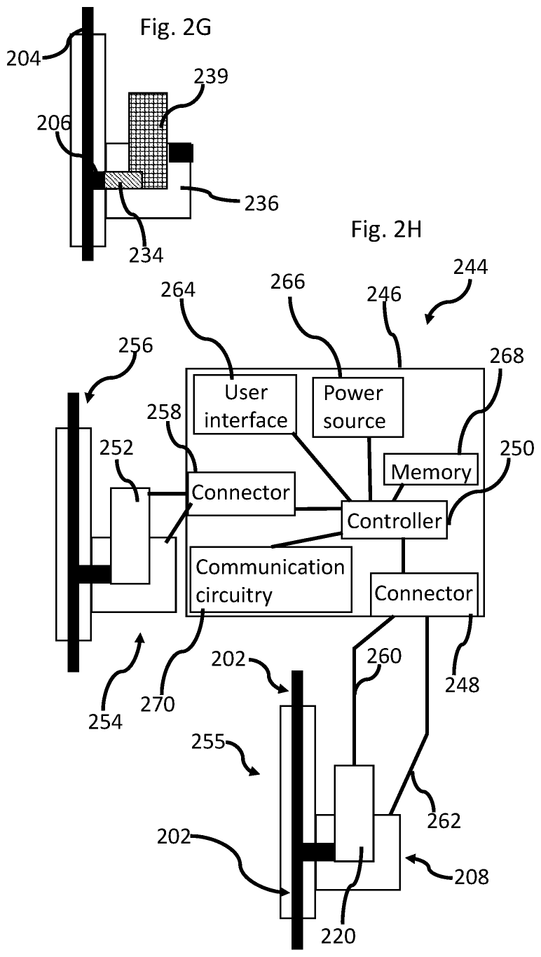

Reference is now made to FIGS. 2A-2B depicting a strut assembly of a strut and a motor adaptor coupled to the strut, according to some exemplary embodiments of the invention.

According to some exemplary embodiments, for example as shown in FIG. 2A, an elongated strut 202, comprises a liner actuator, for example linear actuator 204, of a bone fixation device. In some embodiments, the liner actuator 204 is configured to extend and/or to shorten a length of the strut along longitudinal axis 205. In some embodiments, the linear actuator 204 is axially disposed within the strut 202. In some embodiments, the linear actuator comprises a screw. In some embodiments, the linear actuator 204 comprises a gear 206, configured to rotate the linear actuator 204, for example a screw of the linear actuator 204. In some embodiments, the linear actuator gear 206 comprises a knob, a ring, a cog wheel or any rotating element configured to deliver movement to the linear actuator 204.

According to some exemplary embodiments, a motor adaptor, for example motor adaptor 208 comprises housing 210. In some embodiments, the housing is shaped and sized to connect at least a portion of the strut, for example strut 202. In some embodiments, the housing 210 comprises one or more openings, for example openings 214, shaped and sized to fit at least partly around a portion of the strut, for example strut 202.

According to some exemplary embodiments, strut 204 comprises one or more radially extending portions shaped and sized to interface, for example to interlock, with the motor adaptor 208. In some embodiments, the motor adaptor 208 is connected to the strut 204 by one or more pins and/or screws. In some embodiments, the strut 204 comprises one or more visual indicators, for example a window indicating an extending length of the strut. In some embodiments, the motor adaptor 208, for example housing 210, are shaped not to block the one or more visual indicators when the motor adaptor is coupled to the strut.

According to some exemplary embodiments, the motor adaptor comprises at least one motor fastener, for example motor restrainer 216, configured to allow attachment and detachment of a motor from the motor adaptor. Additionally, the motor restrainer 216 restrains movement of the motor, relative to the strut, when the motor is attached to the motor adaptor. In some embodiments, the motor restrainer 216 is configured to restrain lateral and/or axial movement of the motor relative to the strut.

According to some exemplary embodiments, the motor adaptor 208 comprises a lock 215, for example in the housing 210, configured to allow easy locking and unlocking of the motor from the motor restrainer 216, and/or from the motor adaptor housing 210. In some embodiments, the lock comprises an interference lock, a quick release lock, or a snap lock. In some embodiments, the lock comprises a clip. In some embodiments, the lock is separable from the housing, for example when the motor is not coupled to the motor adaptor.

According to some exemplary embodiments, the lock 215 comprises a motor unit connector configured to connect the motor unit to the housing of the motor adaptor. In some embodiments, the motor connector comprises one or more protrusions configured to fit into openings in the housing of the motor adaptor and to lock the motor connector to said housing. In some embodiments, the motor unit connector is configured to geometrically interlock with the motor adaptor housing and/or with the motor unit. In some embodiments, the motor unit comprises a groove, and the motor connector is shaped and sized to fit into the groove when fastening the motor unit to the motor adaptor housing.

According to some exemplary embodiments, the motor restrainer 216 comprises a manual actuator interface 217, for example a ring, a knob, a cog wheel. In some embodiments, the manual actuator interface 217 is configured to allow rotation of a linear actuator of a strut coupled to the motor adaptor 208.

According to some exemplary embodiments, the motor restrainer 216 comprises a socket, for example socket 218, configured to contact at least a portion of a motor, for example a motor end. In some embodiments, the socket 218 is shaped and sized to fit around the motor end.

According to some exemplary embodiments, for example as shown in FIG. 2B, the motor adaptor 208 is attached to the strut 202, for example to form a strut assembly. In some embodiments, the motor adaptor 208 is coupled to the strut, for example selectively coupled to the strut 202. In some embodiments, the motor adaptor 208 is coupled to the strut 202 for example by one or more connectors and/or fasteners.

According to some exemplary embodiments, when the motor adaptor 208 is coupled to the strut 202, the linear actuator 204 interacts with a gear of the motor adaptor 216. In some embodiments, when the motor adaptor 208 is coupled to the strut 202, the linear actuator 204 interacts with the manual actuator interface 217, optionally via the linear actuator gear 206. In some embodiments, when the motor adaptor 208 is coupled to the strut 202, movement of the manual actuator interface 217, for example rotation of the interface 217, moves the linear actuator 204.

According to some exemplary embodiments, for example as shown in FIG. 2C, a motor unit 220, for example a detachable motor unit, is coupled, for example selectively coupled, to the motor adaptor 208. In some embodiments, the motor 220 is coupled to the motor adaptor 208 via the motor restrainer 216, for example by contacting the socket 218 of the motor restrainer 216. In some embodiments, at least a portion of the motor 220, for example a motor end, is connected to the restrainer 216, for example to the socket 218 of the restrainer 216. In some embodiments, the restrainer 216 restrains movement of the motor 220, for example movement of the motor end, relative to the strut 202.

According to some exemplary embodiments, the motor unit 220 comprises housing, a motor and a gear extending from said motor unit within said housing. In some embodiments, the motor unit end comprises a portion of the motor unit gear extending out from the housing. In some embodiments, the portion of the gear extending from the housing has a smaller diameter compared to a diameter of the motor unit housing.

According to some exemplary embodiments, for example as shown in FIG. 2C, when the motor 220 is coupled to the motor adaptor 208, at least a portion of the motor 220, for example the motor end, interacts with the linear actuator, for example with the gear 206. Optionally, the motor end interlocks with the gear 206.

According to some exemplary embodiments, for example as shown in FIG. 2C, coupling of the motor 220 to the motor adaptor 208, releases the manual actuator interface 217 from the motor adaptor 216. In some embodiments, the motor end when coupled share the same space in the motor adaptor 208, for example in the restrainer 216, causing decoupling of the manual actuator interface 217 from the motor adaptor 208.

According to some exemplary embodiments, for example as shown in FIG. 2D, the motor adaptor comprises a hinge 221 between the restrainer 216 and a portion of the housing 210 coupled to the strut 202. In some embodiments, the hinge is configured to adjust an angle 122 between the motor 220 and the strut 202, while keeping the motor restrained and in an interaction with the linear acturator. In some embodiments, the hinge comprises a hinge lock, configured to lock the motor 220 at a selected angle between the motor 220 and the strut 202. In some embodiments, the hinge 221 is conjured to move the restrainer 216 and/or the motor 220 coupled to the restrainer 216, at an angle in a range of 0-90 degrees, for example 0-25 degrees, 0-45 degrees, 20-50 degrees, 10-80 degrees, or any intermediate, smaller or larger range of values, relative to the strut.

According to some exemplary embodiments, for example as shown in FIG. 2E, a motor adaptor, for example motor adaptor 228 comprises a gear 234 at the housing 230 of the motor adaptor 228. In some embodiments, the gear 234 is located at or near the motor fastener, for example at the restrainer 236. In some embodiments, the gear 234 is positioned at a location in the motor adaptor 228 that allows interaction with a manual motor interface 217 and/or with a motor coupled to the restrainer 236. In some embodiments, the gear 234 of the motor adaptor contacts, for example directly contacts a linear actuator or a gear of the linear actuator.

According to some exemplary embodiments, the motor adaptor gear is configured to deliver movement from a motor coupled to the motor adaptor, for example as shown in FIG. 2F, to the linear actuator, for example via the linear actuator gear 206. In some embodiments, the motor adaptor gear 234 comprises at least one cog wheel. In some embodiments, the motor adaptor gear 234 is configured to interlock with the linear actuator, for example with the linear actuator gear 206. Additionally, the motor adaptor gear 234 is configured to interlock with the manual motor interface 217 and/or with a motor end or with a gear of the motor.

According to some exemplary embodiments, for example as shown in FIG. 2G, a gear lock, for example gear lock 239 is coupled to the motor adaptor, for example to a motor restrainer 236 of the motor adaptor. In some embodiments, the gear lock 239 is attachable and detachable from the motor adaptor. In some embodiments, the gear lock 239 is reversibly coupled to the motor adaptor, for example to the motor restrainer. Optionally, at least a portion of the gear lock 239 is shaped and sized to be positioned within a socket of the motor restrainer. In some embodiments, the gear lock 239 is coupled, for example interlocks with a gear 206 of the linear actuator 204. Alternatively or optionally, the gear lock 239 is coupled, for example interlocks with the motor adaptor gear 234. In some embodiments, the gear lock 239 is configured to prevent movement of the linear actuator 204 and/or movement of the motor adaptor gear 234, when the motor is detached from the motor adaptor.

According to some exemplary embodiments, a portion of the gear lock 239, for example an end of the gear lock 239 extending out from the motor restrainer 236 has a geometrical shape that matches at least part of the motor adaptor housing. In some embodiments, a geometry, for example a non-symmetrical geometry, of the end of the gear lock 239 extending out from the motor restrainer 236 interlocks with the at least part of the motor adaptor housing.

According to some exemplary embodiments, the gear lock 239 comprises a first end shaped and sized to be interlock with the motor adaptor gear 234 and/or with the linear actuator gear 206. In some embodiments, a second end of the gear lock 239, for example an end of the gear lock extending out from the motor restrainer 236, is configured to interlock with a housing of the motor adaptor and/or with the strut. In some embodiments, interlocking of the gear lock 239 with the housing of the motor adaptor housing and/or with the strut while functionally coupling the linear actuator gear and/or the motor adaptor gear, prevents movement of the linear actuator.

### Exemplary System

According to some exemplary embodiments, a control unit is connected to at least some of the strut assemblies, for example to control and/or to monitor the movement of each strut. In some embodiments, monitoring the strut movement, allows for example, to monitor treatment progress and/or to make treatment adjustments. In some embodiments, monitoring the strut movements is performed from a remote location. Reference is now made to FIG. 2H, depicting a control unit connected to one or more strut assemblies, according to some exemplary embodiments of the invention.

According to some exemplary embodiments, a strut assembly, for example strut assembly 255 comprises a strut, for example a strut 202, a motor adaptor 208 coupled to the strut 202 and a motor, for example motor 220 coupled to the motor adaptor 208. In some embodiments, the strut assembly 255 is connected to a control unit 244, for example an interface module, via the motor. Alternatively, the strut assembly 255 is connected to the control unit via the strut adaptor. Some potential control and monitoring processes of a control unit, for example an interface module are described in application WO2017/221243 incorporated herein as a reference in its entirety.

According to some exemplary embodiments, the control unit 244 comprises housing 246, configured to be attached to a bone fixation device, for example to a ring or an arc of a bone fixation device. In some embodiments, the housing 246 is configured to be attached to the bone fixation device via a housing adaptor. In some embodiments, the adaptor is configured to be attached to the bone fixation device, for example by one or more screws or any type of a fastener. In some embodiments, the housing of the control unit is configured to attach and detach from the housing adaptor, for example using an interference lock or a snap fit lock of the adaptor.

According to some exemplary embodiments, each strut assembly, for example strut assembly 255 is connected to the control unit 244 via a strut assembly connector, for example connector 248, located in the housing 246. In some embodiments, the strut assembly 255 is connected to the connector 248 via one or more wires, for example one or more cables. In some embodiments, the cables are electrical cables. In some embodiments, a motor of the strut assembly, for example motor 220 is connected to the connector 248, for example by cable 260. Alternatively or additionally, a motor adaptor 208 is connected to the connector, for example by cable 262. In some embodiments, cables 260 and 262 transmit power and data between the strut assembly 255 and the control unit 244.

According to some exemplary embodiments, the control unit 244 comprises a different connector, for example connector 258 for connecting a different strut assembly, for example strut assembly 256 to the control unit 244. In some embodiments, each of the connectors of the control unit 244 and/or each of the strut assemblies, for example the motors of the strut assemblies, are coded, for example with a visual code. In some embodiments, a connector and a strut assembly, for example a motor of a strut assembly are coded with a matching or a complementary code, for example a numerical code, a color code, a pattern code. In some embodiments, the code allows to connect a specific strut assembly, for example a specific motor, with a specific connector of the control unit. In some embodiments, the code allows to connect a specific motor to a specific strut motor adaptor in a pre-determine order.

According to some exemplary embodiments, the control unit 244 comprises a controller 250, connected to each of the connectors of the control unit, for example connector 248 and 258. In some embodiments, the control unit 244 comprises memory 268, which stores at least one of one or more treatment protocols, values of at least one treatment parameter, log files of the control unit, indications regarding the activation of each of the motors connected to the control unit, and indications regarding the current length of each of the struts. In some embodiments, the at least one parameter comprises an activation parameter of each of the motors, for example activation timing, number of strut extension sessions per hour, per day, per week and/or per month, strut extension length per session, and/or motor activation parameters needed for each strut extension session.

According to some exemplary embodiments, the control unit 244 comprises at least one user interface, for example user interface 264. In some embodiments, the user interface 264 is configured to deliver a human detectable indication, for example a visual indication and/or an audio indication to the patient, to a physician, to a nurse or to a caregiver of the patient. In some embodiments, the controller 250 is configured to monitor the proper connection of the motors to the motor adaptors and/or the proper activation of the motors, by measuring electrical current and/or voltage of the motors.

According to some exemplary embodiments, if one or more of the motors is not connected properly, or is not activated according to a selected treatment plan, the controller 250 signals the user interface to generate a human detectable indication. Alternatively or additionally, if a specific motor is not connected to a predetermined connector of the control unit, the controller 250 signals the user interface 264 to generate a human detectable indication.

According to some exemplary embodiments, if values of at least one electric parameter of a motor is different from a predetermined value or a range of predetermined values, the control system stops the operation of the motor and/or delivers an alert signal. In some embodiments, if current values of a specific motor are higher or lower than a pre-determined value, the control system delivers an alert signal and/or stops the activation of the specific motor and/or stops treatment plan execution. Optionally the control system re-activates a specific non-activated motor in a later time.

According to some exemplary embodiments, the control unit 244 comprises a communication circuitry 270, configured to transmit and receive signals from a remote device, for example a device that is not physically connected to the control unit 244. In some embodiments, the remote device comprises a cellular phone, a wearable device, a remote computer, a tablet, a remote server, an information storage cloud. In some embodiments, the communication circuitry transmits and receives wireless signals, for example Bluetooth signals, Wi-Fi signals, infrared signals or any other wireless signals. According to some exemplary embodiments, the communication circuitry 270 and/or the user interface 264 comprise a memory storage adaptor, for example any type of a Universal Serial Bus (USB) adaptor, for example to allow connection of a memory storage device to the control unit 244.

According to some exemplary embodiments, if the information received from the motors or from the motor adaptors indicate that a motor is not connected properly, or that a treatment plan progress is not as desired, the controller 250 signals the communication circuitry to deliver an indication to a remote device, for example to signal the remote device to generate a human detectable indication.

According to some exemplary embodiments, the control unit 244 comprises a power source 266, for example an electric power source. In some embodiments, the power source comprises a battery, for example a non-replaceable battery or a replaceable battery or a rechargeable battery. In some embodiments, the control unit 244 delivers electric power from the power source 266 to each of the motors via each connector, and the cables connecting the control unit 244 and each motor or each motor adaptor.

According to some exemplary embodiments, the control unit 244 signals a user interface of a strut or a user interface of a motor coupled to the strut, to generate a human detectable indication, for example a visual and/or an audio indication. In some embodiments, the generated indication indicates a current status of the strut or a current status of the motor. In some embodiments, the user interface comprises at least one LED indicator and/or a speaker.

### Exemplary Detailed Process for Motor Coupling

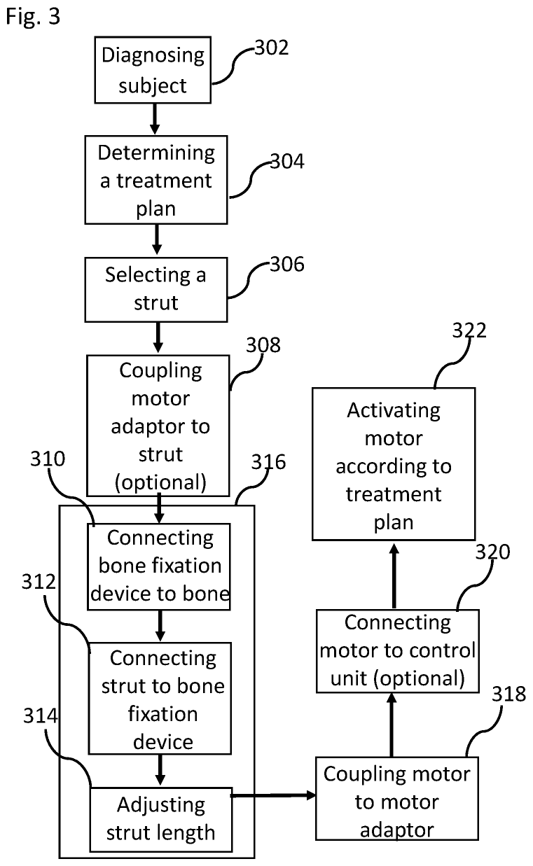

According to some exemplary embodiments, a motor is attached and detached from a strut of a bone fixation device, for example from a motor adaptor of the strut, while the strut remains connected to the bone fixation device. Reference is now made to FIG. 3 depicting a detailed process for coupling, for example selectively coupling, of a motor to a strut, according to some exemplary embodiments of the invention.

According to some exemplary embodiments, a subject is diagnosed at block 302. In some embodiments, the subject is diagnosed with a bone deformation, for example bone fracture. In some embodiments, the subject is diagnosed by performing tissue imaging, for example x-ray, computerized tomography (CT), ultrasound (US) and/or magnetic resonance imaging (MRI).

According to some exemplary embodiments, a treatment plan is determined at block 304. In some embodiments, the treatment plan is determined based on the result of the diagnosis performed at block 302. Additionally or alternatively, the treatment plan is determined based of the age of the patient, the severity of the bone deformation, and the location and/or orientation of the bone parts that need to be fixed to the bone fixation device. In some embodiments, parameters of the determined treatment plan comprise at least one of number of strut extension sessions per day, strut extension length per each extension session, timing of each extension session, and duration of each extension session. In some embodiments, additional parameters comprise the current distance between each bone part, and a desired distance between each bone part at the end of the treatment.

According to some exemplary embodiments, a strut is selected at block 306. In some embodiments, the strut is selected according to the determined treatment plan. In some embodiments, the strut is selected based on current distance between bone parts and/or the desired distance between the bone parts at the end of the treatment. Additionally or alternatively, the strut is selected according to the anatomy of the patient.

Alternatively, a treatment plan is determined after the bone fixation device is attached to the limb, for example in the operating room. In some embodiments, a strut is selected prior to the determining of the treatment plan. Optionally, the determined treatment plan is adjusted during and/or following the attachment of the bone fixation device to the bone of a patient.

According to some exemplary embodiments, a motor adaptor is coupled to the strut at block 308. In some embodiments, the motor adaptor is coupled to the strut outside the operation room, for example during the strut manufacturing process. Alternatively, the motor adaptor is coupled to the strut in the factory, and is provided as a strut assembly comprising the strut and the motor adaptor. In some embodiments, the strut or the motor adaptor are sterilized. Alternatively, the strut and the motor adaptor are sterilized as a single integral unit, for example as the strut assembly.

According to some exemplary embodiments, the bone fixation device is connected to the bone of a patient at block 310. In some embodiments, pins, for example transfixation pins, and/or wires are inserted into the bone. In some embodiments, the pins are connected to an external fixator, for example a frame of a bone fixation device. In some embodiments, the frame comprises a monorail, rod, a closed ring, and open-ring, or an arc-shaped frame.

According to some exemplary embodiments, at least one strut, for example the selected strut is connected between two external fixators, of a bone fixation device. In some embodiments, the at least one strut comprises 2, 3, 4, 5, 6, 7, 8 struts or any larger number of struts.

According to some exemplary embodiments, the bone fixation device is attached to a fractured bone. Alternatively, a fracture is generated after the bone fixation device is attached to a bone.

According to some exemplary embodiments, a length of one or more of the struts is adjusted at block 314, for example during the attachment of the bone fixation device to the bone. In some embodiments, the length of the strut is adjusted to fit between the two external fixators, for example between two frames. Alternatively or additionally, the length of the strut is adjusted according to a predetermined starting point of the treatment. In some embodiments, the length of the one or more struts is adjusted manually, for example by moving a manual interface of the motor adaptor coupled to the strut. In some embodiments, the manual interface is moved, for example rotated using a hand or a digit of a subject, for example a nurse or a physician. Alternatively, the manual interface is moved, for example rotated using a tool inserted into the manual interface, for example a screwdriver, a ratchet, a hex key or any other tool shaped and sized to be placed within the manual interface.

According to some exemplary embodiments, a length of one or more of the struts is adjusted at block 314 while an end of the strut is connected to a first frame. In some embodiments, a length of the one or more of the struts is adjusted to allow connection of the strut to a second frame of the bone fixation device.

According to some exemplary embodiments, at least some or all of the bone fixation connection at block 310, the connection of the strut at block 312 and the adjusting of the strut length, are performed in a surgical operation room, for example as part of a surgical process.

According to some exemplary embodiments, motors coupled to at least some of the struts at block 318. In some embodiments, a different motor is coupled to each strut. In some embodiments, the motor is coupled to a motor adaptor attached to the strut, for example attached to a linear actuator of the strut. In some embodiments, the motor is reversibly coupled to the strut. In some embodiments, a motor is coupled to each of the struts. In some embodiments, a specific motor is coupled to a specific strut, for example based on a predetermined plan.

According to some exemplary embodiments, during and/or following the coupling of the motors, for example motor units, the motors are identified. In some embodiments, the motor units are identified for example to make sure that the correct motors are connected to the correct struts. In some embodiments, each of the motor units comprises a unique identification code, for example a barcode and/or a RFID. In some embodiments, the identification code is read by a computer, for example a barcode reader or a RFID reader, respectively.

According to some exemplary embodiments, at least one motor coupled to the strut is connected to a control unit, for example an interface module, at block 320. In some embodiments, the motor is connected to the control unit prior to the coupling of the motor to the strut. In some embodiments, the control unit comprises the control unit 244 described in FIG. 2H. In some embodiments, the motor is connected to the control unit via the motor adaptor of the strut. In some embodiments, the connection of the motor to the control unit comprises electrical power delivery between the control unit and the motor, and/or information transmission between the control unit and the motor. In some embodiments, each motor coupled to a strut is connected to a different and optionally specific, connector of the control unit, for example as shown in FIG. 2H. In some embodiments, connection of a motor to a wrong connector leads to the generation and delivery of an alert signal, for example a human detectable alert signal.

According to some exemplary embodiments, each of the motors coupled to the struts are activated at block 322. In some embodiments, the motors are activated according to the treatment plan determined at block 304. Alternatively or additionally, the motors are activated according to a predetermined activation plan per each motor. Optionally, the motors are activated in synchronization. In some embodiments, the motors are activated based on signals received from the control unit.

### Exemplary Motor Coupling to a Strut

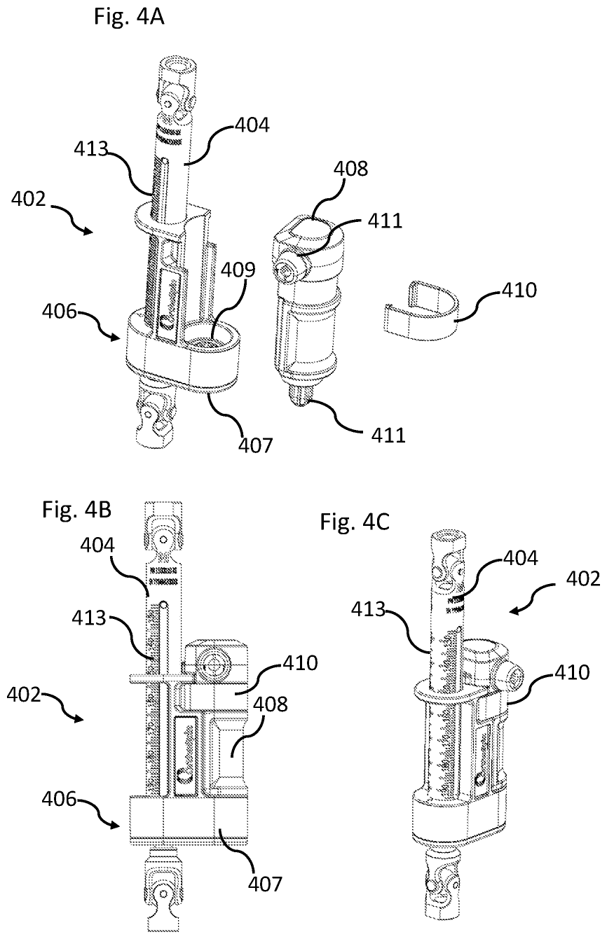

Reference is now made to FIGS. 4A-4C depicting an assembly or a kit, of a strut, a motor adaptor and a motor, according to some exemplary embodiments of the invention.

According to some exemplary embodiments, a strut assembly, for example strut assembly 402 comprises an elongated strut 404 and a motor adaptor 406 coupled to the strut 404. In some embodiments, the motor adaptor 406 is fixedly coupled to the strut 404, for example during the manufacturing process of the strut 404. In some embodiments, the motor adaptor comprises a motor restrainer 407, configured to connect a motor to the motor adaptor, and to restrain movement of the motor relative to the strut. In some embodiments, the motor restrainer comprises a socket 409, shaped and sized to receive at least a portion of a motor, for example an end of the motor.

According to some exemplary embodiments, the strut 404 comprises a linear actuator, for example a linear actuator disposed within the strut 404. In some embodiments, the linear actuator comprises a screw.

According to some exemplary embodiments, a kit comprises the strut assembly 402 and a motor 408, for example an electrical motor. In some embodiments, at least a portion of the motor, for example the motor end 411, is shaped and sized to interact with the motor adaptor, for example to connect to the motor adaptor.

According to some exemplary embodiments, the motor 408 comprises at least one cable connector 411 configured to be connected to a cable, for example an electric cable. In some embodiments, the cable connects the control unit to the motor 408, for example as described in FIG. 2H. In some embodiments, the cable connector, for example cable connector is positioned near an end of the motor which is opposite to the motor end interacting with the motor adaptor. Alternatively or additionally, the cable connector is located at a distance from an extending portion of the strut, for example to ensure that a distance between the motor and the control unit remains constant.

According to some exemplary embodiments, for example as shown in FIGS. 4B and 4C, the cable connector is located at a distance of at least 5 cm, for example 6 cm, 7 cm, 10 cm or any intermediate, smaller or larger value, for moving portions of the motor or the strut. For example to minimize interaction and/or a distance between a cable connected to the cable connector and the moving portions of the motor or strut.

According to some exemplary embodiments, the strut 404 comprises an indicator, for example external indicator 413, configured to provide a visual indication regarding the extension length of the strut. In some embodiments, the indicator comprises a ruler.

According to some exemplary embodiments, the kit comprises one or more motor fasteners, for example fastener 410, configured to fasten and/or lock the motor to the strut and/or to the motor adaptor housing. In some embodiments, the one or more fasteners comprise a clip, a band, or an elastic band.

According to some exemplary embodiments, for example as shown in FIGS. 4B and 4C, once coupled to the motor adaptor, the assembly of the strut, the motor adaptor and the motor are fastened together in a way that prevents unwanted decoupling of the motor and/or the motor adaptor from the strut, for example during treatment.

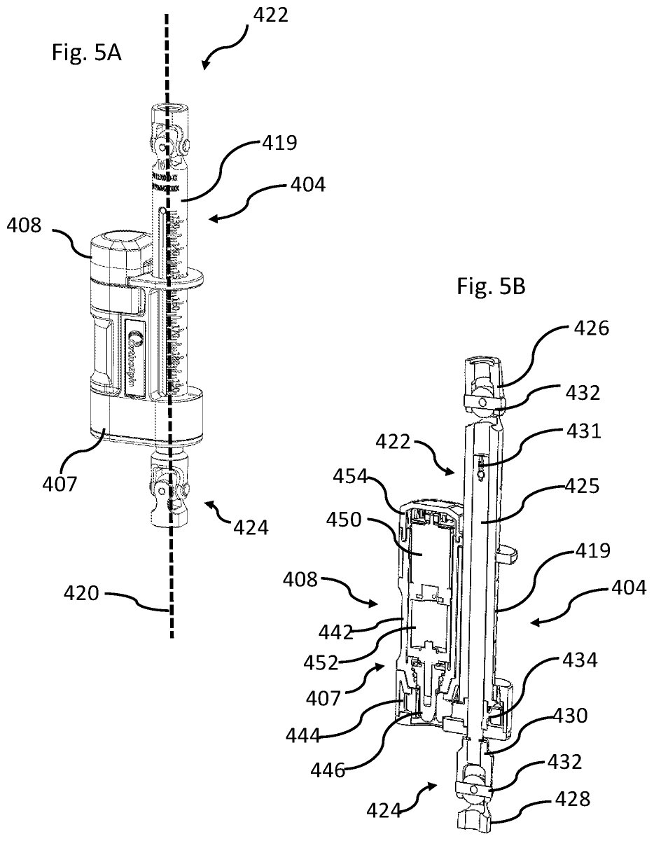

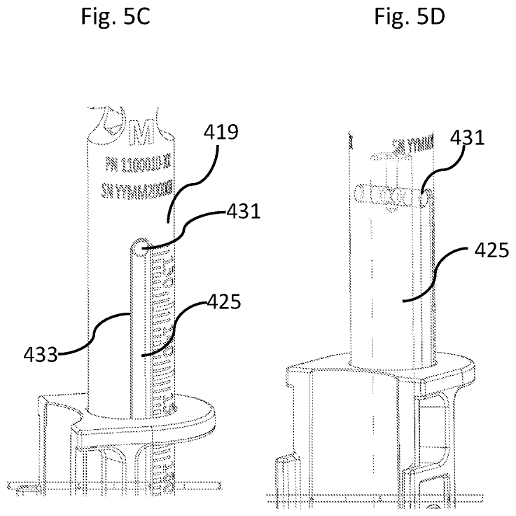

Reference is now made to FIGS. 5A and 5B, depicting assembly components and interaction between the motor, the motor adaptor and the strut, according to some exemplary embodiments of the invention.

According to some exemplary embodiments, the strut 404 comprises an elongated body 419 having a longitudinal axis 420, a first end 422 and a second end 424. In some embodiments, the first end 422 of the strut is a stationary end, configured not to move relative to the strut body 419. In some embodiments, the second end 424 of the strut 404 is a moving end, for example an extending end, configured to move relative to the strut body 404. In some embodiments, the motor adaptor comprises one or more openings that allow passing of the strut body. In some embodiments, the openings in the motor adaptor are round and are configured to rotate the motor adaptor around the strut body.

According to some exemplary embodiments, for example as shown in FIG. 5B, the strut 404 comprises a linear actuator disposed within the strut body, for example a strut screw 425. In some embodiments, the strut comprises at least two connectors, for example mechanical connectors, each at a different end of the strut. In some embodiments, at least one connector, for example a joint 426, is connected to the body 419 of the strut 404 at the stationary end 422. In some embodiments, at least one different connector, for example a joint 428 is connected to an extending portion 430 of the strut screw 425, at an extending end 424 of the strut 404. In some embodiments, the joint 426 and/or joint 428 are external fixation ring joints, for example M7 or M5 ring joints. Optionally, one or more of the joints comprise a ball, for example a titanium ball 432. In some embodiments, the at least two connectors of the strut, for example joints 426 and/or 426 are configured to connect the strut to bone fixation device frames, for example rings. In some embodiments, the strut is coupled to two spaced apart frames of a bone fixation device using the joints, where each joint connects a different end of the strut to a different frame.

According to some exemplary embodiments, the strut 404 comprises a linear actuator transmission member, for example a strut gear 434. In some embodiments, the strut gear 434 is located at a distance of less than 5 cm, for example less than 4 cm, less than 2 cm, less than 1 cm, or any intermediate, smaller or larger distance from the extending portion 430 of the strut. In some embodiments, the strut gear is coupled, for example fixedly coupled to the linear actuator 425. In some embodiments, at least a portion of the external surface of the linear actuator comprises threading. In some embodiments, the strut gear 434 is coupled to the threading of the linear actuator 425, for example interlock with the threading of the linear actuator 425. In some embodiments, the strut gear 434 comprises a screw nut which interlock with the threading, for example a spiral threading around the linear actuator external surface. In some embodiments, the linear actuator is shaped as a cylinder having an external threading along at least 50%, for example at least 70%, at least 80% or any intermediate, smaller or larger percentage value of the linear actuator length.

According to some exemplary embodiments, a motor adaptor 407 comprises housing 442, which is attached at least partly around the strut 404, for example around the strut gear 434. In some embodiments, the motor adaptor 407 comprises a motor adaptor gear 444 in the housing 442. In some embodiments, the motor adaptor gear 444, comprises a cog wheel. In some embodiments, the motor adaptor gear 444 interlocks with the strut gear 434, when the motor adaptor 407 is coupled to the strut 404.

According to some exemplary embodiments, the motor adaptor comprises a motor fastener 446, for example motor restrainer, in the housing 442, configured to receive at least part of a motor and to restrain movements, for example restrain lateral and/or axial movements of the motor part relative to the strut. In some embodiments, the motor fastener comprises a socket, which is shaped and sized to fit an end, for example a rotating end of the motor. In some embodiments, the socket is coupled to the motor adaptor gear 444, and is configured to transmit rotation movement of the motor end to the motor adaptor gear 444, while optionally restraining the movement of the motor end, for example during rotation of the motor end.

According to some exemplary embodiments, for example as shown in FIG. 5B, rotation power is transmitted to the linear actuator 425 from the motor 408 near the extending portion 430 of the strut 404.

According to some exemplary embodiments, for example as shown in FIG. 5B, the motor comprises an electric motor 450, for example a direct current (DC) motor, connected to a motor gear 452. In some embodiments, the motor gear shaft is coupled to the motor fastener 446, for example to a socket of the motor fastener 446. In some embodiments, coupling of the motor gear shaft to the motor fastener 446, allows, for example engagement with the motor adaptor gear 444 that is coupled with the linear actuator gear 434.

According to some exemplary embodiments, the motor 407 comprises at least one positioning sensor, for example positioning sensor 454. In some embodiments, the positioning sensor is configured to monitor the extension length of the strut based on the motor rotational movement.

According to some exemplary embodiments, for example as shown in FIGS. 5B-5D, a pin 431 crossing through linear actuator 425, and at least one slit 433 in the body 419, prevents rotation of the linear actuator 425 relative to the body 419, as gear 434 rotates.

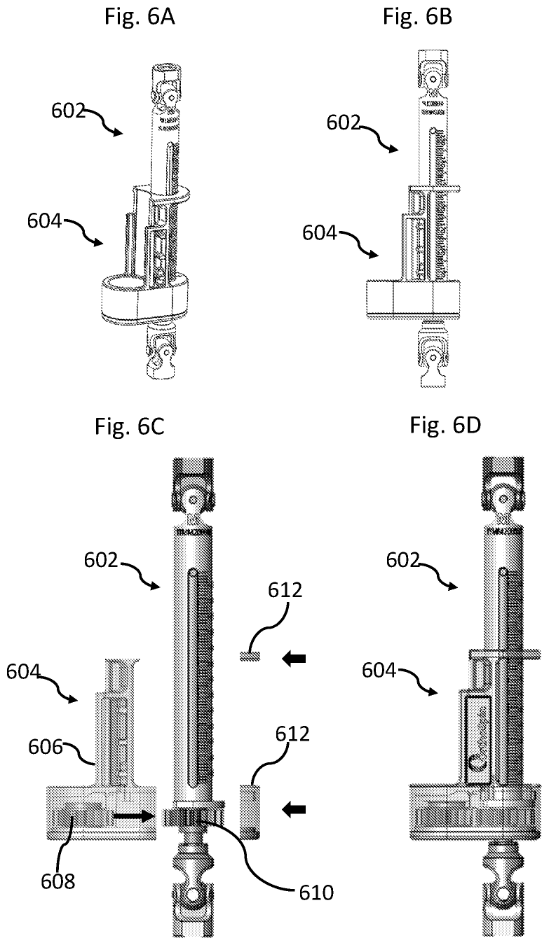

Reference is now made to FIGS. 6A-6D depicting an add-on motor adaptor selectively coupled to a strut, according to some exemplary embodiments of the invention.

According to some exemplary embodiments, a motor adaptor, for example motor adaptor 604 is configured to be selectively coupled to a strut, for example strut 602. In some embodiments, the motor adaptor 604 comprises a transmission member, for example a gear 608. In some embodiments, the gear 608 comprises a cog wheel. In some embodiments, a housing 606 of the motor adaptor 604 is shaped and sized to align and attach the gear 608 to a strut linear actuator gear 610, for example to align and interlock the gear 608 of the motor adaptor 604 with the strut gear 608. In some embodiments, one the gear 608 interlocks with the linear actuator gear 610, one or more fasteners, for example fasteners 612, lock a position of the motor adaptor 604 relative to the strut 602. In some embodiments, the one or more fasteners 612 comprise a clip, or a band. Alternatively, the one or more fasteners comprise a part of the housing 606 configured to be removed to allow attachment of the motor adaptor 604 to the strut 602, and to be re-joined to the housing 606 for fixedly attaching the motor adaptor 604 to the strut 602, for example as shown in FIG. 6D.

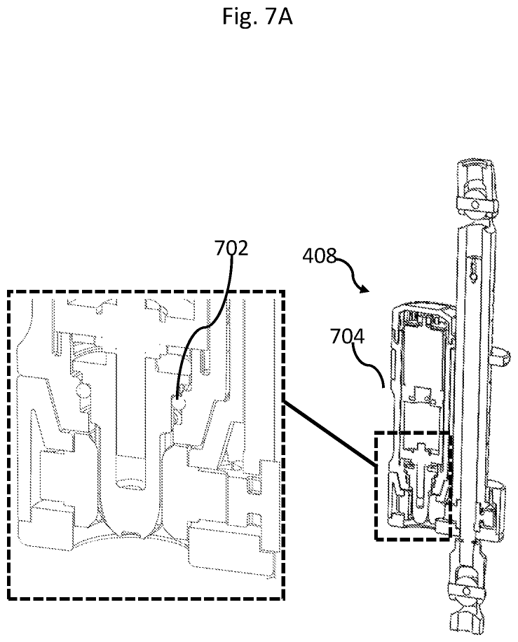

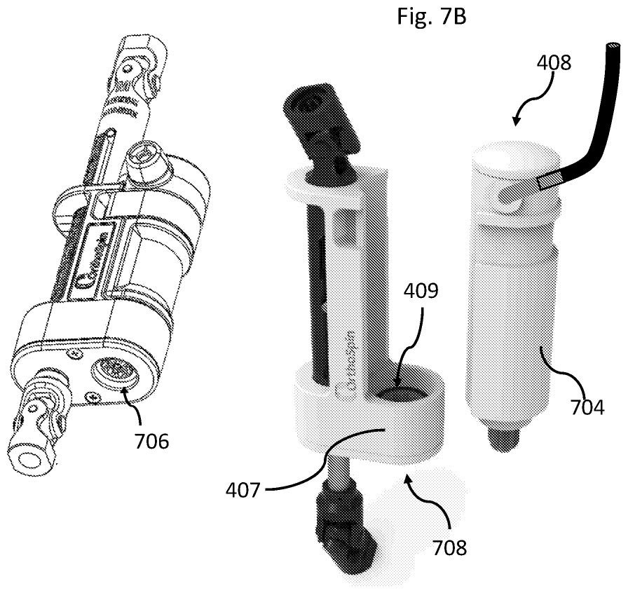

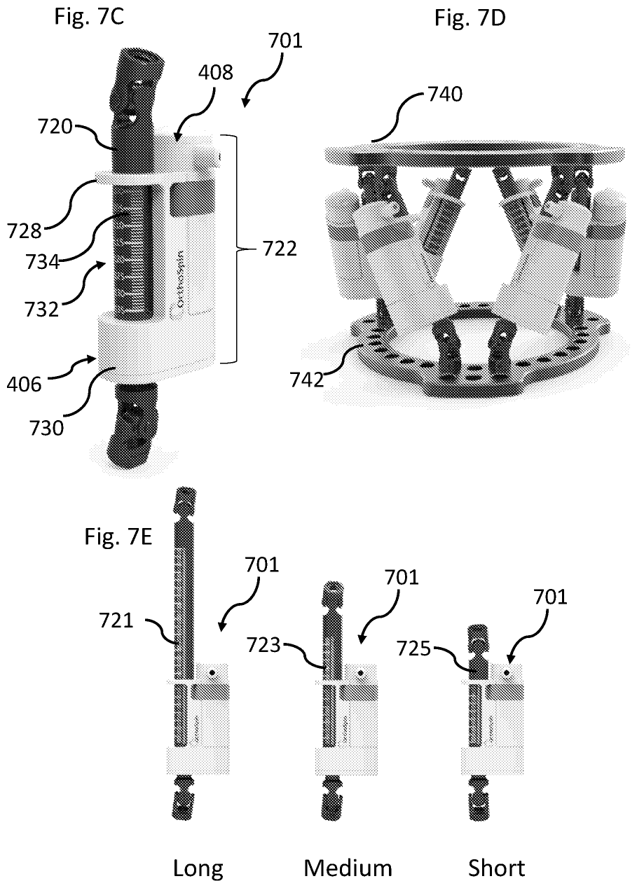

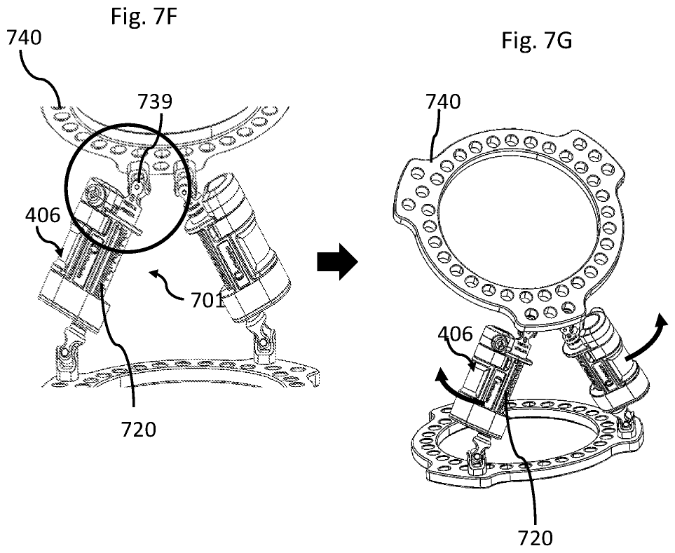

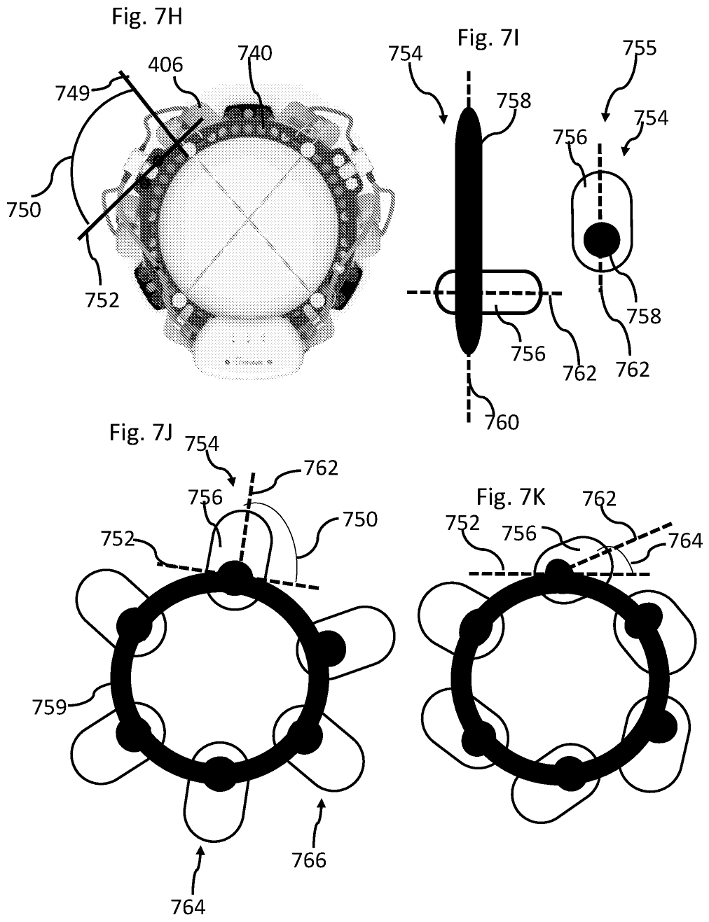

According to some exemplary embodiments, for example as shown in FIG. 7A, the motor 408 is sealed against penetration of water. In some embodiments, the motor 408 comprises a seal 702. In some embodiments, the seal is positioned between a rotating end of the motor, extending out from the motor housing 704 and an inner lumen of the housing. In some embodiments, sealing the motor against water allows at least IP67 water protection, for example to allow a patient to which the bone fixation device is mounted to submerge the bone fixation device in water, for example during water rehabilitation treatments.

According to some exemplary embodiments, additionally, for example as shown in FIG. 7B, the external surface 704 of the motor 408 is smooth, for example to allow easy wiping of the surface, for example opening 706. In some embodiments, sealing the motor against water allows, for example easy maintenance and cleaning of the motor.

According to some exemplary embodiments, for example as shown in FIG. 7B, the motor fastener 407, for example a socket 409 of the motor fastener comprises at least one drainage hole 706, for example to allow water drainage from the socket 409.

According to some exemplary embodiments, for example as shown in FIGS. 7C and 7D, an assembly 701 between the motor adaptor 406 and the motor 408, fits around a small strut, for example strut 720. In some embodiments, a length of the assembly is shorter than a length between two ends of the strut 720. In some embodiments, a maximal length of the assembly when the linear actuator is at minimum length is in a range of 6 cm to 25 cm, for example 6 cm-8 cm, 10 cm-12 cm, 18 cm-20 cm or any intermediate, shorter or longer assembly length.

According to some exemplary embodiments, for example as shown in FIG. 7C, the housing of the motor adaptor 406 comprises an opening 732 or a window, which is aligned with an indicator, for example ruler 734 of the strut, when the motor adaptor 406 is attached to the strut 720. In some embodiments, the opening 732 allows to visualize an indicator, indicating an extension length of the strut. In some embodiments, the opening is positioned between two or more connection points of the motor adaptor to the strut, for example connection points 728 and 730.

According to some exemplary embodiments, for example as shown in FIG. 7D, strut assembly 701, is attached to bone fixation devices rings, for example rings 740 and 742. In some embodiments, the rings have a diameter in a range of 80 mm-300 mm, for example 80 mm-120 mm, 100 mm-150 mm, 130 mm-200 mm, 190 mm-250 mm, 200 mm-300 mm or any intermediate, smaller or larger range of values. Optionally, the assembly 701 can fit struts of bone fixation devices where an angle between the rings is up to 60 degrees, for example up to 55 degrees, up to 50 degrees or any intermediate, smaller or larger angle between the two rings. Optionally, the assembly 701 can fit struts connected closer to the inner diameter of the rings.

According to some exemplary embodiments, for example as shown in FIG. 7E, the assembly 701 is shaped and sized to be attached to struts with different lengths, for example by one or more motor adaptor housing openings and/or one or more connectors of the motor adaptor housing. In some embodiments, one or more openings of the motor adaptor housing have an inner diameter which is larger than the external diameter of the strut. In some embodiments, the inner diameter of the one or more openings is larger in up to 3 mm, for example up to 2 mm, up to 1 mm, or any intermediate, smaller or larger value from the external diameter of the strut. In some embodiments, the one or more motor adaptor openings form a channel shaped to receive the strut.

According to some exemplary embodiments, the assembly 701 is shaped and sized to be attached to a long strut 721, for example a strut that has a minimal length in a closed state in a range of 160 mm-190 mm, for example 160 mm-170 mm, 165 mm-180 mm, 175 mm-190 mm or any intermediate, smaller or larger range of values. In some embodiments, the assembly 701 is shaped and sized to be attached to a medium strut 723, for example a strut that has a minimal length in a closed state in a range of 110 mm-130 mm, for example 110 mm-120 mm, 115 mm-130 mm or any intermediate, smaller or larger range of values. In some embodiments, the assembly 701 is shaped and sized to be attached to a short strut 725, for example a strut that has a minimal length in a closed state in a range of 80 mm-110 mm, for example 80 mm-100 mm, 90 mm-100 mm, 95 mm-110 mm or any intermediate, smaller or larger range of values. In some embodiments, different assemblies are used with different strut sizes.

### Changing an Angle Between a Motor Adaptor and a Bone Fixation Device

According to some exemplary embodiments, a motor adaptor coupled to a strut is configured to rotate around an axis of the strut, for example to adjust an angle between the motor adaptor and the bone fixation device, for example between the motor adaptor and at least one frame of the bone fixation device. In some embodiments, the angle is adjusted between a motor adaptor coupled to the strut and at least one frame connected to the strut. In some embodiments, an angle between the motor adaptor and the bone fixation device is changed, for example, to reduce a portion of the motor adaptor extending out from a bone fixation device perimeter. Reference is now made to FIGS. 7F-7K depicting adjusting an angle between a motor adaptor and a bone fixation device, for example a frame of the bone fixation device, according to some exemplary embodiments of the invention.

According to some exemplary embodiments, for example as shown in FIGS. 7F and 7G, a motor adaptor 406 connected to strut 720, is configured to rotate around an axis of the strut, for example to change an angle between the motor adaptor and a frame of a bone fixation device, for example frame 740. In some embodiments, a rotation lock 739, for example a locking pin, in the strut is released, to allow rotation of the strut 720 a round a longitudinal axis of the strut. In some embodiments, rotation of the strut 720 rotates the motor adaptor 406 coupled to the strut 720. In some embodiments, when reaching a desired angle between the motor adaptor 406 and the bone fixation device, for example the frame 740, the rotation lock 739 is locked to prevent undesired rotation of the motor adaptor. As used herein, rotation of the motor adaptor refers to rotation of a strut assembly comprising a motor adaptor and a strut around a longitudinal axis of the strut.

According to some exemplary embodiments, for example as shown in FIG. 7H, an angle 750 between 750 between a transverse axis 749 of the motor adaptor 406 or a strut assembly, and a frame 740 of a bone fixation device, for example a tangent 752 of the frame 740, is in a range between 0-90 degrees, for example 0-30 degrees, 20-40 degrees, 50-90 degrees or any intermediate, smaller or larger range of angle values.

According to some exemplary embodiments, for example as shown in FIG. 7I, a strut assembly 754 comprises a motor adaptor 756 coupled to a strut 758. In some embodiments, the strut 758 comprises an elongated body having a longitudinal axis 760. In some embodiments, a motor adaptor 756 coupled to the strut 758 has a transverse plane or a horizontal axis 762, which is optionally perpendicular to the longitudinal axis 760. FIG. 7I depicts a cross-section 755 along horizontal axis 762.

According to some exemplary embodiments, for example as shown in FIG. 7J, at least one ring of a bone fixation device, for example ring 759 is connected to two or more struts, for example strut assemblies. In some embodiments, a motor adaptor 756 of a strut assembly 754 or the strut assembly 754 as a single unit is rotated around the axis 760, and locked at angle 750 between a horizontal axis 756, and a tangent 752, for example a tangent plane perpendicular to the ring 759. In some embodiments, the angle 750 is in a range of 0-90 degrees. In some embodiments, for example as shown in FIG. 7J, the angle is 90 degrees. In some embodiments, when the angle is 90 degrees, the motor adaptor maximally extends from a bone fixation device perimeter.

According to some exemplary embodiments, for example as shown in FIG. 7K, at least some of the motor adaptors, or the strut assemblies are rotated, for example to reduce a portion of the motor adaptor extending from the bone fixation device perimeter. In some embodiments, reducing an extending portion of the motor adaptor allows, for example, to prevent contact between external objects in the surroundings of the patient with one or more of, the motor adaptor, a motor coupled to the motor adaptor, at least one cable connecting the motor or the motor adaptor to a control unit.

According to some exemplary embodiments, for example as shown in FIG. 7K, the motor adaptor 756, or the strut assembly including the motor adaptor, is locked at angle 764 which is smaller than 90 degrees, for example, smaller than 45 degrees, smaller than 30 degrees, smaller than 10 degrees, smaller than 5 degrees.

### Exemplary Strut Replacement During Treatment

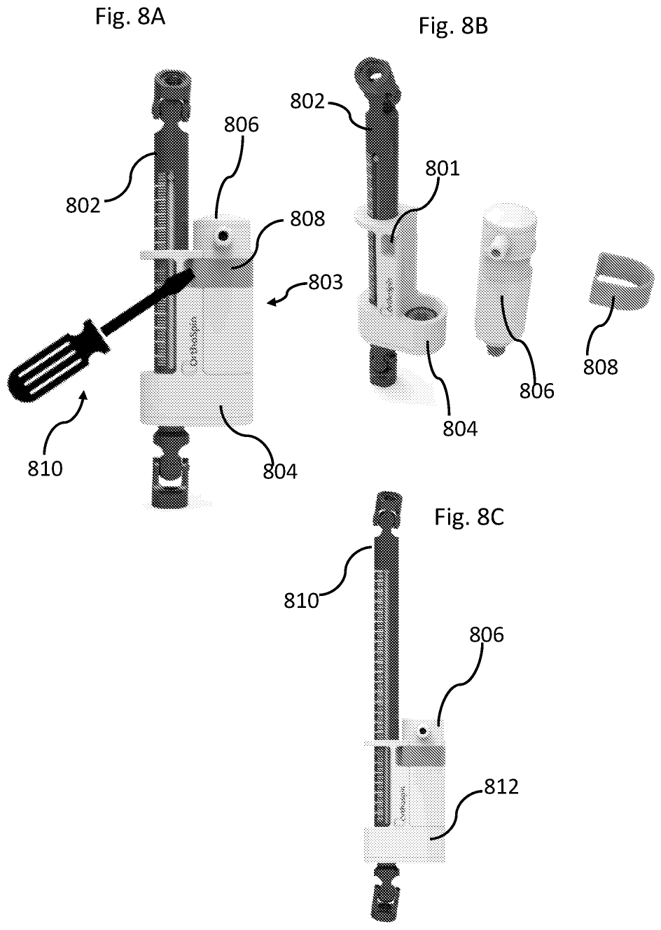

According to some exemplary embodiments, there is a need to replace a strut during a treatment. Reference is now made to FIGS. 8A-8C depicting replacement of a strut during a treatment, according to some exemplary embodiments of the invention.

According to some exemplary embodiments, for example as shown in FIG. 8A, a motor 806 is detached from a motor adaptor 804, which is coupled to strut 802. In some embodiments, the motor 806 is detached from the motor adaptor 804 by releasing at least one fastener, for example fastener 808 fastening the motor 806 to the motor adaptor 804. In some embodiments, the fastener comprises a clip configured to be attached to openings 801 in the motor adaptor housing. In some embodiments, the fastener 808 is released using a tool 810. In some embodiments, the tool 810 has a unique geometrical or structural shape to allow, for example, releasing of the fastener 808. In some embodiments, using a tool with a unique geometry allows to prevent unwanted removal of the motors by the patient. In some embodiments, the motor is removed in the clinic, or in a medical facility, for example when replacing the strut.

According to some exemplary embodiments, for example as shown in FIG. 8B, once the motor 806 is removed from the motor adaptor 804, the strut 802 with the motor adaptor is replaced. Alternatively, the motor adaptor 804 is released from the strut 802 and only the strut 802 is replaced.

According to some exemplary embodiments, for example as shown in FIG. 8C, once the strut and the motor adaptor are replaced with strut 810 and motor adaptor 812, the motor 806 used with the previous strut is coupled to the motor adaptor 812.

According to some exemplary embodiments, the motor 806 is selectively coupled to the motor adaptor without using a tool, for example by placing the fastener, for example an elastic clip around the motor and within the openings 801. In some embodiments, releasing the fastener 808 from the openings requires a tool, for example to prevent an unwanted release of the motors during treatment.

### Exemplary Manual Strut Adjustment

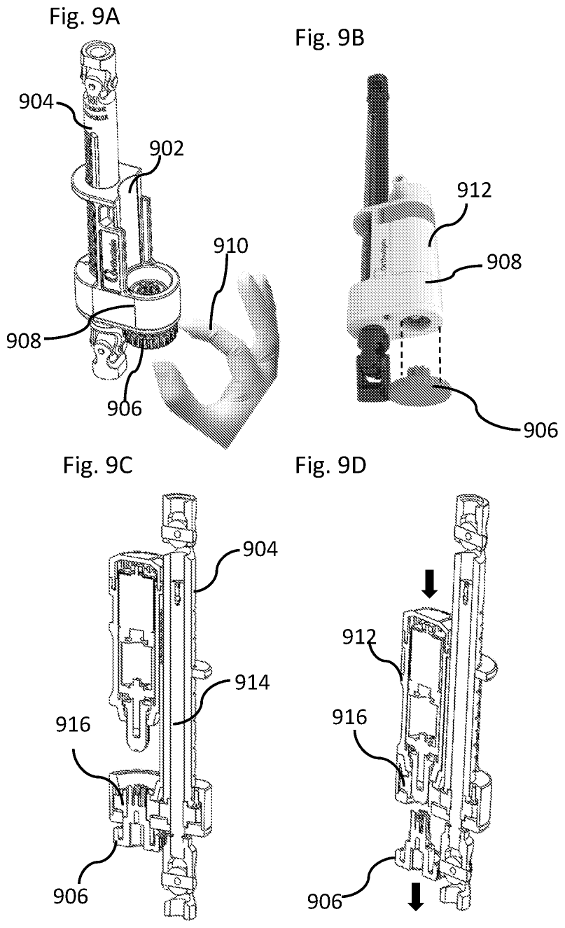

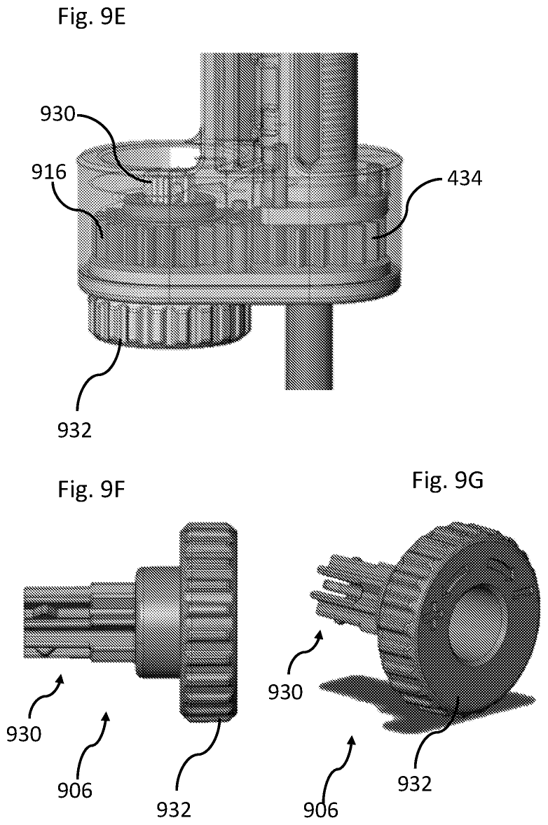

According to some exemplary embodiments, a motor adaptor coupled to a strut is configured to allow manual movement of a linear actuator of the strut, for example during strut adjustments and/or calibration. Reference is now made to FIGS. 9A-9G, depicting a motor adaptor manual interface, according to some exemplary embodiments of the invention.

According to some exemplary embodiments, a motor adaptor 902 coupled to strut 904 comprises a motor adaptor manual interface, for example manual interface 906. In some embodiments, a motor fastener of the motor adaptor, for example motor restrainer 908 comprises the manual interface 906. In some embodiments, the manual interface 906 comprises an inner portion 930 shaped and sized to be positioned at least partly within the motor restrainer 908, for example within a socket of the motor restrainer. Additionally, an external portion 932 of the manual interface 906 is configured to allow manual movement of the manual interface.

According to some exemplary embodiments, the external portion 932 of the manual interface 906, for example an external portion 932 of the manual interface 906 extending out from the motor adaptor, as shown in FIG. 9E, is shaped to allow rotation of the manual interface, for example by a hand of a user. In some embodiments, the external portion 932 of the manual interface is round, and optionally include a plurality of bulges or protrusions shaped to increase friction with the user hand.

According to some exemplary embodiments, an inner portion 930 of the manual interface 906, is configured to contact a gear, for example gear 916 of the motor adaptor 902. In some embodiments, the manual interface 906, for example the inner portion 930, interlocks with the gear 916, for example as shown in FIG. 9E.

According to some exemplary embodiments, for example as shown in FIGS. 9B and 9D, coupling of a motor, for example motor 912, to the motor adaptor 902, disengages the manual interface 906 from the motor adaptor 902. In some embodiments, the manual interface 906 and the motor 912, for example an end of the motor 912, interchangeably couple the restrainer 908, for example the gear 916. In some embodiments, the manual interface 906 and to the motor 912, for example the motor end, interchangeably interlock with the gear 916.

### Exemplary System Assembly Process

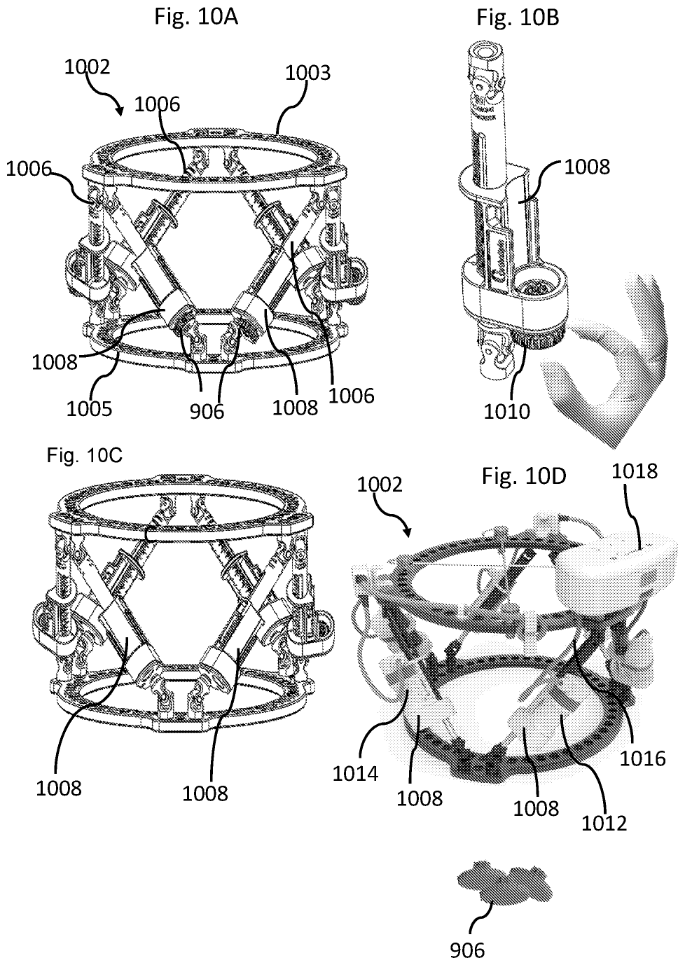

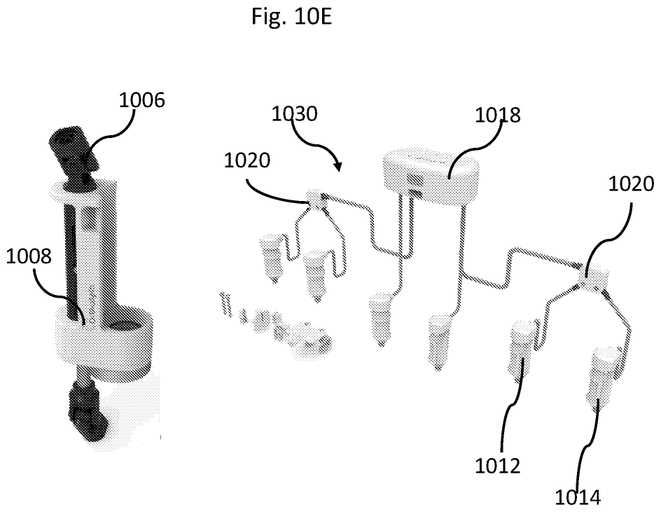

According to some exemplary embodiments, motors are coupled to a bone fixation device, for example to struts of the bone fixation device after a surgery for fixing the bone fixation device to the bone is completed. In some embodiments, the motors are coupled to the struts outside the operating room, for example at a clinic. In some embodiments, coupling the motors separately from the surgery, outside the operating room allows, for example, to sterilize only the struts with the motor adaptors, without a need to sterilize the motors. Additionally or alternatively, coupling the motors separately from the surgery, outside the operating room, allows for example to shorten the time needed in the operating room. Reference is now made to FIGS. 10A-10D, depicting an assembly process of a bone fixation system, according to some exemplary embodiments of the invention.

According to some exemplary embodiments, for example as shown in FIGS. 10A and 10B, a bone fixation device 1002 is connected to a bone during surgery in an operating room. In some embodiments, the bone fixation device 1002 comprises struts for example struts 1006 interconnecting two frames 1003 and 1005 of the bone fixation device. In some embodiments, the frames are closed frames, for example closed circular frames. Alternatively, the frames are open frames, for example arc-shaped frames. Alternatively, the frames comprise any plate or bar connected to a bone pin or nail extending from the bone.

According to some exemplary embodiments, each of the struts 1006 comprise a motor adaptor 1008, coupled to a linear actuator of the strut. In some embodiments, in the operating room, each motor adaptor comprises or at least some of the motor adaptors comprise a manual interface 906. In some embodiments, for example as shown in FIG. 10B, in the operating room, an expert, for example a surgeon, a physician or a nurse, manually adjusts the length of each strut using the manual interface 906, for example as described in FIGS. 9A-9D. In some embodiments, a length of each strut is adjusted in the operating room, according to a distance between the two frames, and an orientation of the frames relative to each other.

According to some exemplary embodiments, for example as shown in FIGS. 10C and 10D, outside the operating room, for example at a clinic or in the patient home, motors are coupled to the motor adaptors, for example motors 1012 and 1014. In some embodiments, coupling of the motors disengages the manual interface 906 from each motor adaptor. Additionally, an interface module, for example a control unit 1018 is attached to the bone fixation device, for example to at least one frame of the bone fixation device. Additionally, the control unit is connected, for example electrically connected to each of the motors via at least one cable for example cable 1016 connecting motor 1012 to the control unit 1018.

According to some exemplary embodiments, each of the motors comprise a visual code that is used for motor identification, for example as a motor ID code. In some embodiments, the motor ID code allows, for example to position the motors in a predetermined location and order, optionally in the different motor adaptors. In some embodiments, the control unit 1018 monitors and/or adjusts the operation of a specific motor using the motor ID code. In some embodiments, after connecting the control unit 1018 to the motors, and optionally the cables to the external fixation device parts, at least one treatment program stored in the memory of the control unit is initiated. Alternatively, at least one of a treatment program is loaded to the control unit memory, for example from an external device, for example an external computer, a remote device, a mobile device. In some embodiments, the activation parameters of one or more of the motors are loaded into a memory of the control unit, for example memory 268 shown in FIG. 2H. In some embodiments, the control unit 1018 activates each of the motors separately and/or in synchronization according to information stored in the memory.

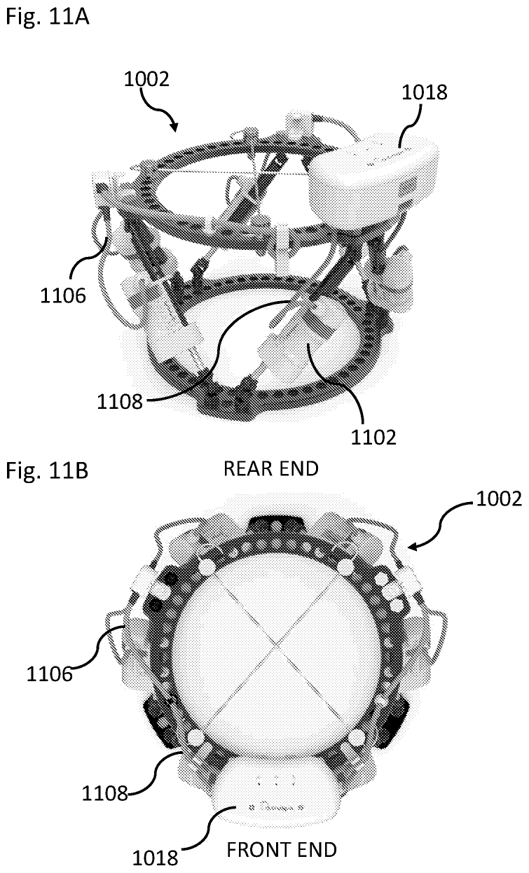

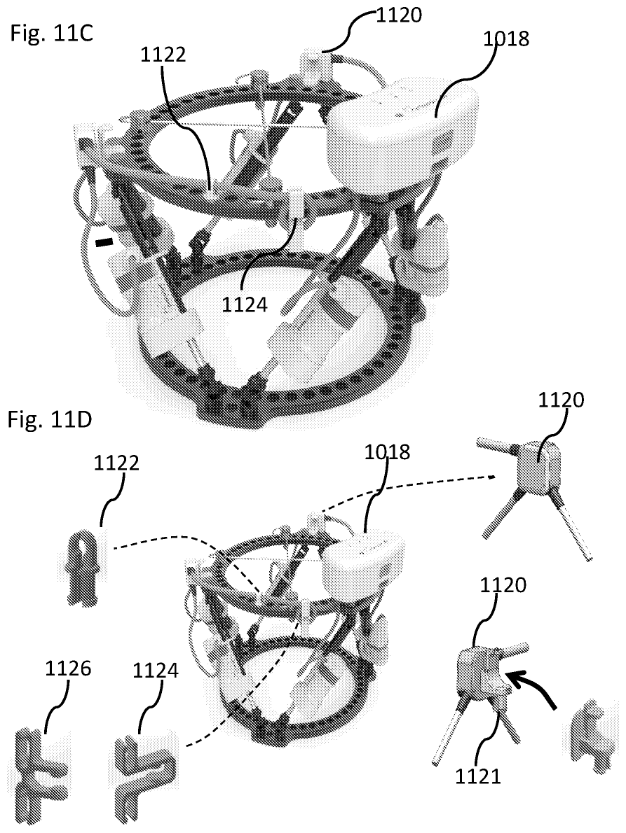

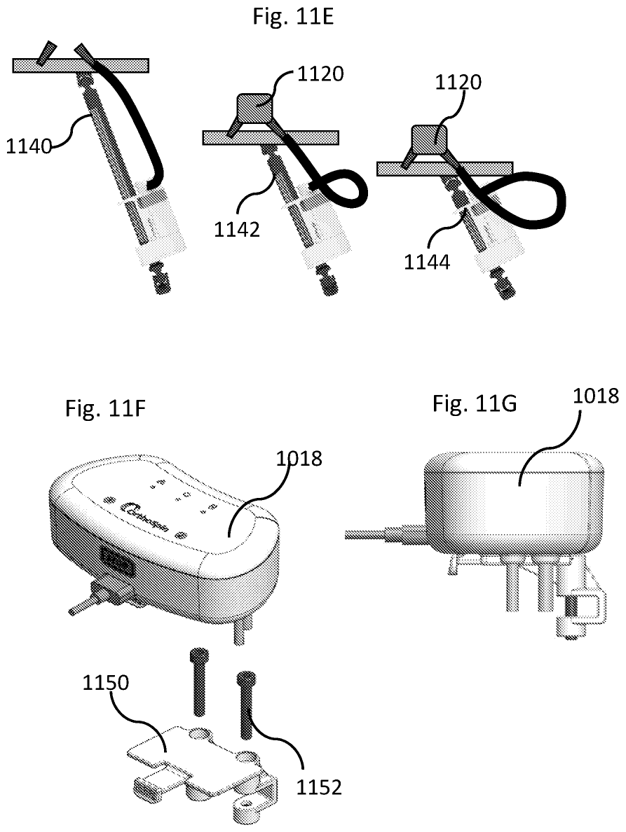

According to some exemplary embodiments, for example as shown in FIG. 10E, coupling the motors and connecting the control unit outside the operating room, allows, for example to sterilize only mechanical components of the bone fixation device, for example the motor adaptor 1008 and the strut 1006, while keeping the control system 1030, comprising the control unit 1014 and two or more motors, for example the motors 1012 and 1014 non sterile. In some embodiments, the strut and the motor adaptor are configured to be sterilized using an autoclave. In some embodiments, for example as shown in FIG. 10E, two or more motors, for example motors 1012 and 1014 are connected via a cable splitter box 1020 to a control unit 1018. Exemplary system assembly on bone fixation device According to some exemplary embodiments, a system for monitoring and/or controlling a bone fixation device is mounted on a bone fixation device in a way that allows an easy installation using a single hand. Additionally, the control system is positioned within a point of view of a patient or a caregiver, for example to allow visualization of one or more indicators on the control unit by the patient and/or caregiver. Additionally or optionally, the control system is positioned in a way that minimizes interference to the system components by external objects surrounding the patient. Reference is now made to FIGS. 11A-11G depicting system assembly onto a bone fixation device, according to some exemplary embodiments of the invention.

According to some exemplary embodiments, for example as shown in FIGS. 11A and 11B, the control unit 1018 is mounted on a front end of the bone fixation device, optionally on the most upper frame of the bone fixation device. Additionally, a panel of the control unit 1018, for example an upper panel, which includes one or more visual indicators is oriented or tilted to face the eyes of the patient.

According to some exemplary embodiments, the system is shaped and sized and/or is mounted on the bone fixation device 1002, not to extend out from the bone fixation 1002 in more than 7 cm, for example more than 5 cm, more than 3 cm or any intermediate, smaller or large value. In some embodiments, cables, for example cables connecting the motors with the control unit 1018 are attached to the bone fixation device, for example to minimize the extension of the cables beyond the bone fixation device perimeter. Additionally or alternatively, the cables are directed towards the rear end of the bone fixation device.

According to some exemplary embodiments, for example as shown in FIGS. 11C and 11D, cables from two or more motors are connected via a cable splitter box, for example box 1120. In some embodiments, for example as shown in FIG. 11C, the box 1120 is attached to a frame of the bone fixation device. In some embodiments, the box 1120 is attached to one or more openings in the frame by an attachment pin 1121 of the box 1120.

According to some exemplary embodiments, for example as shown in FIGS. 11C and 11D, cables are fastened to the bone fixation device, for example to the bone fixation device by one or more cable fasteners 1122. In some embodiments, the one or more cable fasteners are introducible through one or more openings in the bone fixation device, for example openings in a frame of the bone fixation device. In some embodiments, in order to prevent an excess length of a cable to be loose, one or more of the cables is wrapped around a cable wrapper, for example an internal cable wrapper 1126 or an external cable wrapper 1124, attached to the bone fixation device, for example to a frame of the bone fixation device. In some embodiments, in case a cable is loose, for example when using a small external fixation ring, a cable wrapper 1124 or 1126 is used to allow wrapping of the loose cable around the cable wrapper 1124 or 1126.

According to some exemplary embodiments, for example as shown in FIG. 11E, a length of a cable between a motor and a box 1120 is adjusted to fit a long strut, for example long strut 1140, a medium strut 1142 and a small strut 1144.

According to some exemplary embodiments, for example as shown in FIGS. 11F and 11G, a control unit 1018 is attachable and detachable from a bone fixation device, for example from a frame of the bone fixation device. In some embodiments, a control unit ring interface 1150 is fixedly attached to the frame using one or more screws 1152. In some embodiments, the control unit is configured couple, for example to be attached to the ring interface 1150 via at least one quick release lock, for example a snap lock or any interference lock that is configured easily lock and release the control unit 1014 from the ring interface 1150. In some embodiments, the quick release lock is part of the ring interface 1150 and/or the control unit 1018.

### Exemplary Positioning Sensor

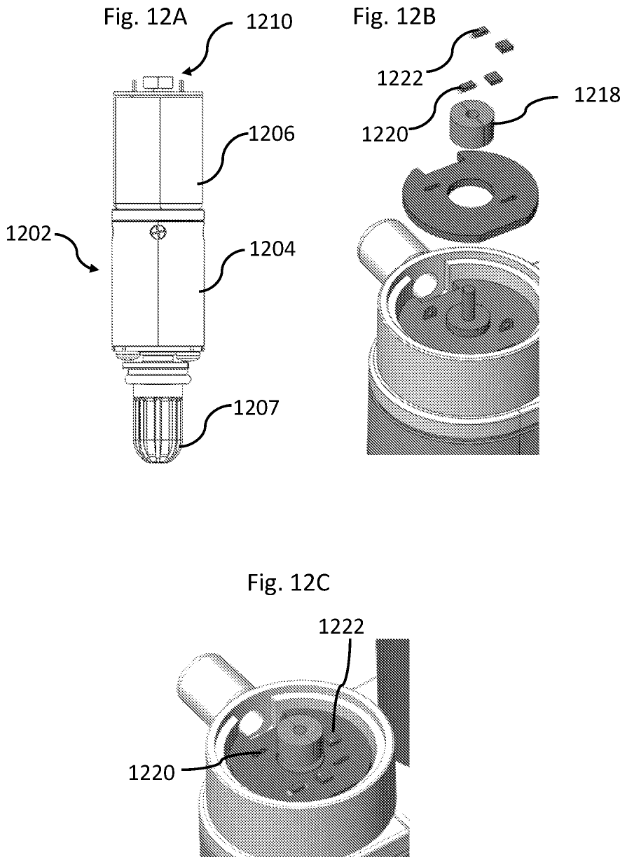

According to some exemplary embodiments, the control unit connected to each of the motors monitors the axial extension length of each of the struts of a bone fixation device, for example by measuring a rotational positioning of each motor. Reference is now made to FIGS. 12A-12C depicting a motor positioning sensor, according to some exemplary embodiments of the invention.

According to some exemplary embodiments, a motor 1202 of a bone fixation device, comprises a gear 1204 coupled, for example axially coupled to a motor 1206, for example a DC motor. In some embodiments, the gear 1204 rotates a motor end 1207 configured to interact, for example to interlock with a gear of a motor adaptor. In some embodiments, the motor 1202 comprises at least one positioning sensor 1210.

According to some exemplary embodiments, the positioning sensor is configured to record the rotation of the motor, at least 3 times, for example at least 4, at least 5 or any smaller or larger number of readings, during a turn of the motor 1206. In some embodiments, a control unit connected to the motor measures an axial positioning of the strut based on the positioning sensor readings. In some embodiments, the control unit measures that axial positioning of the strut with a resolution of at least 0.3 μm, for example 0.5 μm, 0.6 μm or any intermediate, smaller or larger value. In some embodiments, the positioning sensor comprises a rotating magnet 1218 and one or more Hall sensors, for example sensors 1220 and 1222. In some embodiments, the positioning sensor comprises 2, 3, 4, 5, 6, 7, 8 or any larger number of Hall sensors.

### Exemplary Gear Lock

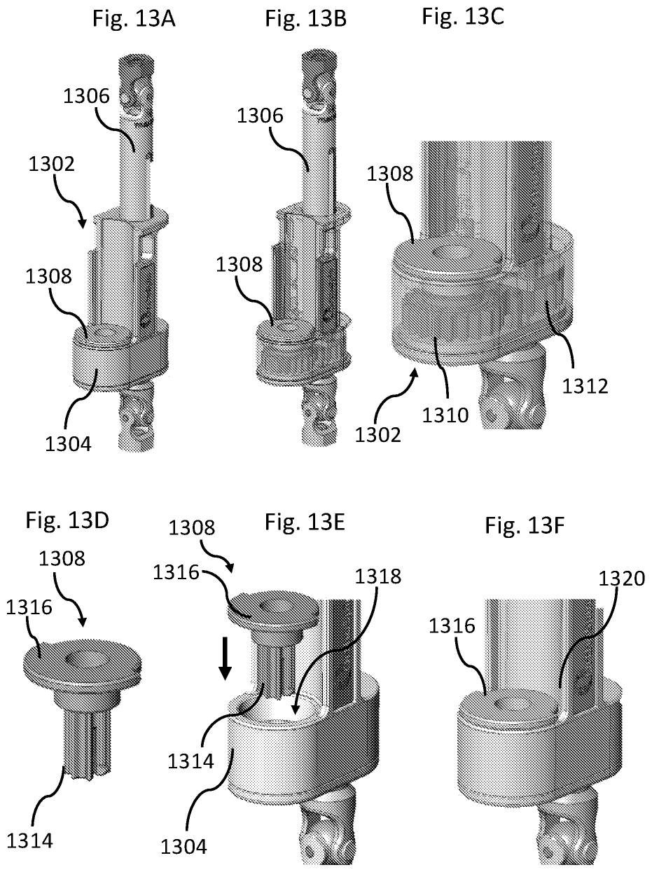

According to some exemplary embodiments, a gear lock is attached to a motor adaptor coupled to a strut, when a motor is snot coupled o the motor adaptor, for example to prevent movement of a linear actuator of a strut. Reference is now made to FIGS. 13A-13F, depicting a gear lock and interaction of the gear lock and a motor adaptor, according to some exemplary embodiments of the invention.

According to some exemplary embodiments, a motor adaptor 1302 is coupled to a strut 1306. In some embodiments, the motor adaptor, comprises a motor restrainer 1304 which is shaped and sized to receive and restrain a portion of a motor unit. In some embodiments, for example when a motor unit is detached from the motor adaptor 1302, a gear lock 1308 is coupled to the motor restrainer 1304. In some embodiments, coupling of the gear lock 1308 to the motor restrainer 1304 prevents movement, for example reversal movement or collapse of a linear actuator of the strut 1306.

According to some exemplary embodiments, for example as shown in FIGS. 13B and 13C, at least a portion of the gear lock 1308, is inserted into the motor restrainer 1304, and interacts with a gear 1310 of the motor adaptor. Optionally, the gear lock 1308, for example a portion of the gear lock 1308 positioned within the motor restrainer 1304 interlocks with the gear 1310. In some embodiments, interlocking of the gear lock 1308 with the gear 1310 prevents movement of the strut linear actuator, for example movement of a linear actuator gear 1312 coupled to the motor adaptor gear 1310.

According to some exemplary embodiments, for example as shown in FIG. 13D, a gear lock 1308 comprises a first end 1314 shaped and sized to fit into a motor restrainer 1304, for example into a socket 1318 of the motor restrainer 1304. In some embodiments, a maximal width of the firs end is smaller than an inner width or an inner diameter of the socket 1318. Additionally or alternatively, the first end I shaped and sized to interlock with a gear 1310, for example with a cog wheel of the gear 1310. In some embodiments, the first end of the gear lock include one or more bulges or protrusions configured to penetrate into openings, for example complementary or matching openings, in the gear 1310, for example in the cog wheel of gear 1310.

According to some exemplary embodiments, a second end 1316 of the gear lock 1308 extends out from the motor restrainer 1304, for example from a socket 1318 of the motor restrainer 1304. In some embodiments, the second end 1316 of the gear lock 1308 is shaped and sized to interact, for example interlock with a housing of the motor adaptor, for example housing 1320. In some embodiments, the gear lock 1308 is configured to interlock with the motor adaptor gear 1310 and the motor adaptor housing 1320 simultaneously, for example to prevent movement, for example rotation movement of the motor adaptor gear 1310.

According to some exemplary embodiments, the second end 1316 of the gear lock 1308 has a geometrical shape configured to interlock with at least one protrusion or a geometrical shape of the housing 1320. In some embodiments, the second end 1316 interlocks with a geometrical shape of the housing, for example a complementary geometrical shape of the housing, for example to prevent movement of the gear lock 1308 relative to the housing.

It is expected that during the life of a patent maturing from this application many relevant struts and bone fixation devices will be developed; the scope of the terms strut and bone fixation device is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-50. (canceled)

51. A bone fixation device, comprising:
a pair of frames having at least one interposed strut having an adjustable length; and
a motor adaptor connected to the strut, the motor adaptor comprising:
a socket for receiving a detachable motor; and
a gear disposed adjacent to the socket, wherein actuation of the gear adjusts the length of the strut.

52. The bone fixation device of claim 51, further comprising a knob for manually engaging the gear and adjusting the length of the strut.

53. The bone fixation device of claim 52, wherein insertion of the detachable motor into the socket disengages the knob.

54. The bone fixation device of claim 51, further comprising a clip for engaging the detachable motor and retaining it in the socket.

55. The bone fixation device of claim 51, wherein the strut defines a longitudinal axis, the strut rotates around its longitudinal axis as the length of the strut is adjusted, and the motor adaptor does not rotate with the strut.

56. The bone fixation device of claim 51, wherein the motor adaptor cooperates with an indicator of the strut for indicating the length of the strut.

57. A bone fixation device, comprising:
a pair of frames having at least one interposed strut, wherein rotation of the strut adjusts the length of the strut; and
a motor adaptor connected to the strut and comprising an actuator for adjusting the length of the strut, wherein the motor adaptor does not rotate with the strut.

58. The bone fixation device of claim 57, further comprising a knob for manually engaging the actuator and adjusting the length of the strut.

59. The bone fixation device of claim 58, wherein the motor adaptor further comprises a socket for receiving a detachable motor, the actuator being disposed adjacent to the socket, wherein insertion of the detachable motor into the socket disengages the knob.

60. The bone fixation device of claim 57, further comprising:
a detachable motor;
a clip for engaging the detachable motor attaching the detachable motor to the motor adaptor.

61. The bone fixation device of claim 60, wherein the motor has an associated radio-frequency identification (RFID).

62. The bone fixation device of claim 60, further comprising a control unit attached to one of the frames and connected to the detachable motor by a cable, wherein the control unit controls the motor to adjust the length of the strut.

63. The bone fixation device of claim 62, wherein the motor has an associated motor ID code, and the control unit uses the motor ID code when controlling the motor.

64. The bone fixation device of claim 62, further comprising a cable clip for connecting the cable to one of the frames.

65. The bone fixation device of claim 62, further comprising a memory, wherein the control unit controls the motor to adjust the length of the strut according to a treatment plan stored in the memory.

66. The bone fixation device of claim 62, further comprising a battery for energizing the control unit and the motor.

67. A bone fixation device, comprising:
a mechanical group comprising:
a pair of frames having at least one interposed strut having an adjustable length, wherein rotation of the strut adjusts a length of the strut; and
a motor adaptor connected to the strut and comprising an actuator for adjusting the length of the strut, wherein the motor adaptor does not rotate with the strut; and
an electrical group comprising:
a detachable motor for being received in the motor adaptor and engaging the actuator;
a control unit attached to one of the frames and connected to the detachable motor by a cable; and
a memory, wherein the control unit controls the motor to adjust the length of the strut according to a treatment plan stored in the memory.

68. The bone fixation device of claim 67, wherein the mechanical group is sterilized before affixation to a patient in need thereof and the electrical group is not sterilized.

69. The bone fixation device of claim 67, wherein the control unit is configured to transmit and receive signals from a remote device, wherein the treatment plan is loaded to the memory from the remote device.

70. The bone fixation device of claim 67, wherein the motor is sealed against penetration of water so that the bone fixation device may be submerged in water.
